(19) United States
(12) Patent Application Publication
(10) Pub. No.: US 2008/0039420 A1
(43) Pub. Date: Feb. 14, 2008

(54) Method for inhibiting NOX1 gene expression

(75) Inventors: Agnes Juhasz, Glendora, CA (US); James H. Doroshow, Bethesda, MD (US); Susan Markel, Pomona, CA (US); Shikha Gaur, Glendora, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 11/785,419

(22) Filed: Apr. 17, 2007

Related U.S. Application Data
(60) Provisional application No. 60/793,242, filed on Apr. 20, 2006.

(51) Int. Cl. A61K 31/7052; A61P 43/00; C07H 21/02; C12N 15/63; C12N 15/85

(52) U.S. Cl. 514/44; 435/320.1; 435/455; 435/466; 536/23.1

(57) ABSTRACT

The present invention relates to compositions and methods for gene-specific inhibition of gene expression by short interfering ribonucleic acid (siRNA) effector molecules. The compositions and methods are particularly useful in modulating gene expression of the NOX1 gene in colon cancer cells.

### CROSS-REFERENCE TO RELATED APPLICATION

The present application is related to and claims priority under 35 U.S.C. §119(e) to U.S. provisional patent application Ser. No. 60/793,242 filed on 20 Apr. 2006, incorporated herein by reference.

### FIELD OF THE INVENTION

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

The present invention relates to compositions and methods for gene-specific inhibition of gene expression by short interfering ribonucleic acid (siRNA) effector molecules. The compositions and methods are particularly useful in modulating gene expression of the NOX1 gene in colon cancer cells.

### BACKGROUND OF THE INVENTION

Reactive oxygen species (ROS) including superoxide, hydrogen peroxide, hydroxyl radicals and their reaction products have been described as damaging agents, which cause DNA mutations, protein oxidations, and lipid peroxidation. Recent studies show the importance of low levels of ROS in cell growth, cell signaling, inflammatory processes as well as cancer development (Droge, 2002; Arnold et al., 2001); Lassegue et al., 2001; Wingler et al., 2001; Arbiser et al., 2002). ROS is produced in response to activation of various cell surface receptors, intracellular signaling and metabolic changes. Receptor-mediated ROS production has been studied extensively in phagocytic cells (Lambeth, 2002; Lambeth, 2004; Lambeth et al., 2000).

In activated phagocytes a NADPH oxidase produces high levels of O₂⁻ as part of microbial host defense. This well characterized enzyme has 2 membrane bound subunits, gp91^{phox} and p22^{phox} which forms a heterodimer and four cytosolic components: p67^{phox}, p47^{phox}, p40^{phox}, and Rac1 or Rac2 (Babior, 1999). Many other cell types including a number of cancer cells have low level of superoxide (Szatrowski and Nathan, 1991). More recently six homologues of gp91^{phox} (renamed as NOX2) were cloned from various non-phagocytic cells; NOX1 (Suh et al., 1999; Banfi et al., 2000; Kikuchi et al., 2000), NOX3 (Cheng et al., 2001), NOX4 (Geiszt et al., 2000), NOX5 (Cheng et al., 2001; Banfi et al., 2001), DUOX1 and DUOX2 (Dupuy et al., 1999).

NOX1, cloned from CaCo2 colon cancer cells (Suh et al., 1999; Dupuy, 2002), is primarily expressed in colon tissue with low level expression in prostate, uterus and vascular smooth muscle cells. The NOX1 catalytic subunit contains binding sites for FAD, NADPH and heme, and the N-terminal portion contains six hydrophobic segments, which form transmembrane a helices (Lambeth et al., 2000; Banfi et al., 2000). NOX1 gp91phox homolog subunit in association with the another transmembrane localized subunit p22^{phox} functions as a superoxide producing enzyme only when co-expressed with its soluble subunits; p47^{phox} homolog NOX1 organizer (NOXO1) and p67^{phox} homolog NOX1 activator (NOXA1) (Banfi et al., 2003; Takeya et al., 2003; Cheng and Lambeth, 2004; Ambasta et al., 2004). The enzyme oxidizes NADPH on the cytosolic side of the membrane, transfers the electron across the membrane to reduce oxygen to superoxide (O₂⁻) which is converted to H₂O₂ in a secondary reaction (Lambeth, 2004).

It has been shown that overexpression of NOX1 results in increased ROS generation, which was partially reversed by the addition of catalase, a H₂O₂ scavenger (Arnold et al., 2001; Suh et al., 1999). Platelet-derived growth factor (PDGF) induced NOXI expression and superoxide production in NIH 3T3 cells stably transfected with NOX1. These transfected cells caused aggressive tumor growth in athymic mice (Suh et al., 1999). It was implicated that PDGF increased NOX1 expression in smooth muscle cells (Lassegue et al., 2001). Lim et. al. showed a correlation over-expression of NOX1 and increased ROS production with metastatic potential in prostate cancer cells (Lim et al., 2005).

Recently, tissue expression of NOX1 in normal and colon cancer at different stage were studied by immunohistochemistry, in situ hybridization and real-time PCR. It has been reported that NOX1 protein showed differentiation-dependent expression in adenocarcinomas. They found that NF-κB protein is co-expressed with NOX1 suggesting that NOX1 overexpression might be associated with NF-κK-dependent pathways in colon adenocarcinomas (Fukuyama et al., 2005 ). Recent studies inhibiting NOX1 with siRNA indicate that decreased NOX1 expression interacts with the Ras/MAPK and phosphatidylinositol 3-kinase (PI3K) pathways (Park et al., 2004; Misushita et al., 2004).

RNA interference (RNAi) is a process in which double stranded RNA (ds RNA) induces the postranscriptional degradation of homologous transcripts, and has been observed in a variety of organisms including plants, fungi, insects, protozans, and mammals (Moss et al., 2001; Bernstein et al., 2001; Elbashir, et al., 2001a, 2001b). RNAi is initiated by exposing cells to dsRNA either via transfection or endogenous expression. Double-stranded RNAs are processed into 21 to 23 nucleotide (nt) fragments known as siRNA (small interfering RNAs) (Elbashir et al., 2001a, 2001b). These siRNAs form a complex known as the RNA Induced Silencing Complex or RISC (Bernstein et al., 2001; Hammond et al. 2001), which functions in homologous target RNA destruction. In mammalian systems, the sequence specific RNAi effect can be observed by introduction of siRNAs either via transfection or endogenous expression of 21-23 base transcripts or longer hairpin precursors. Use of siRNAs evades the dsRNA induced interferon and PKR pathways that lead to non-specific inhibition of gene expression. (Elbashir et al., 2001a).

The discovery of siRNAs permitted RNAi to be used as an experimental tool in higher eukaryotes. Typically, siRNAs are chemically synthesized as 21mers with a central 19 bp duplex region and symmetric 2-base 3′-overhangs on the termini. These duplexes are transfected into cells lines, directly mimicking the products made by Dicer in vivo. Most siRNA sequences can be administered to cultured cells or to animals without eliciting an interferon response (Heidel et la., 2004; Ma et al., 2005; Judge et al., 2005). There are some reports that particular motifs can induce such a response when delivered via lipids (Judge et al., 2005; Sledz et al., 2003; Hornung et al., 2005), although a cyclodextrin-containing polycation system has been shown to deliver siRNA containing one such putative immunostimulatory motif that achieves target gene down-regulation in mice without triggering an interferon response (Hu-Lieskovan et al., 2005), even in a disseminated tumor model.

It has been recently described that chemically synthesized RNA duplexes of 25-30 base length can have as much as a 100-fold increase in potency compared with 21mers at the same location. At the site most extensively examined in this study, EGFPS1, only minor differences in potency were seen between duplexes with blunt, 3′-overhang or 5′-overhang ends, and a blunt 27mer duplex was most potent (Kim et al., 2005). Increased potency has similarly been described for 29mer stem short hairpin RNAs (shRNAs) when compared with 19mer stem hairpins (Siolas et al., 2005). While the primary function of Dicer is generally thought to be cleavage of long substrate dsRNAs into short siRNA products, Dicer also introduces the cleaved siRNA duplexes into nascent RISC in Drosophila (Lee et al., 2004); Pham et al., 2004; Tomari et al., 2004). Dicer is involved in RISC assembly and is itself part of the pre-RISC complex (Sontheimer et al., 2005). The observed increased potency obtained using longer RNAs in triggering RNAi is theorized to result from providing Dicer with a substrate (27mer) instead of a product (21mer) and that this improves the rate or efficiency of entry of the siRNA duplex into RISC.

Unfortunately, not all 27mers show this kind of increased potency. It is well known that shifting a 21 mer siRNA by a few bases along the mRNA sequence can change its potency by 10-fold or more (Holen et al., 2002); Harborth et al., 2003; Reynolds et al., 2004). Different products that result from dicing can have different functional potency, and control of the dicing reaction may be necessary to best utilize Dicer-substrate RNAs in RNAi. The EGFPSI blunt 27mer studied in Kim et al. (2005) is diced into two distinct 21mers. Vermeulen and colleagues reported studies where synthetic 61mer duplex RNAs were digested using recombinant human Dicer in vitro and examined for cut sites using a ³²P-end-labeled gel assay system. Heterogeneous cleavage patterns were observed and the presence of blunt versus 3′-overhang ends altered precise cleavage sites (Vermeulen et al., 2005). Dicing patterns for short 25-30mer RNA substrates have not been published and processing rules that enable accurate prediction of these patterns do not exist. Dicing patterns were studied at a variety of sites using different duplex designs to see if cleavage products could be predicted. It has been found that a wide variety of dicing patterns can result from blunt 27mer duplexes. An asymmetric duplex having a single 2-base 3′-overhang generally has a more predictable and limited dicing pattern where a major cleavage site is located 21-22 bases from the overhang. Including DNA residues at the 3′ end of the blunt side of an asymmetric duplex further limits heterogeneity in dicing patterns and makes it possible to design 27mer duplexes that result in predictable products after dicing.

It has been found that position of the 3′-overhang influences potency and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript. Novel designs described here that incorporate a combination of asymmetric 3′-overhang with DNA residues in the blunt end offer a reliable approach to design Dicer-substrate RNA duplexes for use in RNAi applications. See also U.S. published application Nos. 2005/0244858 A1 and 2005/0277610 A1, each incorporated herein by reference.

Recently, several groups have demonstrated that siRNAs can be effectively transcribed by Pol III promoters in human cells and elicit target specific mRNA degradation. (Lee et al., 2002; Miyagishi et al., 2002; Paul et al., 2002; Brummelkamp et al., 2002; Ketting et al., 2001). These siRNA encoding genes have been transiently transfected into human cells using plasmid or episomal viral backbones for delivery. Transient siRNA expression can be useful for rapid phenotypic determinations preliminary to making constructs designed to obtain long term siRNA expression. Of particular interest is the fact that not all sites along a given mRNA are equally sensitive to siRNA mediated downregulation. (Elbashir et al., 2001a; Lee et al., 2001; Yu et al., 2002; Holen et al., 2002).

In contrast to post-transcriptional silencing involving degradation of mRNA by short siRNAs, the use of long siRNAs to methylate DNA has been shown to provide an alternate means of gene silencing in plants. (Hamilton et al., 2002). In higher order eukaryotes, DNA is methylated at cytosines located 5′ to guanosine in the CpG dinucleotide. This modification has important regulatory effects on gene expression, especially when involving CpG-rich areas known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosomes of females. Aberrant methylation of normally unmethylated CpG islands has been documented as a relatively frequent event in immortalized and transformed cells and has been associated with transcriptional inactivation of defined tumor suppressor genes in human cancers. In this last situation, promoter region hypermethylation stands as an alternative to coding region mutations in eliminating tumor suppression gene function. (Herman et al., 1996). The use of siRNAs for directing methylation of a target gene is described in U.S. published application No. 2004/0171118 A1, incorporated herein by reference.

U.S. published application No. 2004/0096843 A1, incorporated herein by reference, is directed to methods for producing double-stranded, interfering RNA molecules in mammalian cells. These methods overcome prior limitations to the use of siRNA as a therapeutic agent in vertebrate cells, including the need for short, highly defined RNAs to be delivered to target cells other than through the use of synthetic, duplexed RNAs delivered exogenously to cells. U.S. published application No. 2004/0091918 A1, incorporated herein by reference, is directed to methods and kits for synthesis of siRNA expression kits.

It is desired to silence NOX1 expression in HT-29 human colon cancer cells and demonstrate subsequent changes in ROS production, cell growth, cell cycle regulation and gene expression in both cells and corresponding xenografts.

### SUMMARY OF THE INVENTION

The present invention relates to compositions and methods for gene-specific inhibition of gene expression by short interfering ribonucleic acid (siRNA) effector molecules. The compositions and methods are particularly useful in modulating gene expression of the NOX1 gene in colon cancer cells.

Thus, in a first aspect, the present invention provides siRNA molecules, each comprising a sense strand and an antisense strand, wherein the antisense strand has a sequence sufficiently complementary to a NOS1 mRNA sequence to direct target-specific RNA interference (RNAi).

In a second aspect, the present invention provides pharmaceutical compositions containing the disclosed siRNA molecules.

In another aspect, the invention relates to a method for down-regulating and/or inhibiting expression of NOS1 in a colon cancer cell, such as mammalian colon cancer cell, including a human colon cancer cell. The method comprising introducing into a colon cancer cell the disclosed siRNA molecules in an amount sufficient for degradation of target mRNA to occur, thereby activating target-specific RNAi in the cell. The introduction into the cell may be by: (a) contacting the cell with the siRNA; (b) contacting the cell with a composition comprising the siRNA and a lipophillic carrier; (c) transfecting or infecting the cell with a vector comprising nucleic acid sequences capable of producing the siRNA when transcribed in the cell; (d) injecting into the cell a vector comprising nucleic acid sequences capable of producing the siRNA when transcribed in the cell, as well as other techniques well known to a skilled artisan.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIGS. 1A and 1B show the expression of NOX1 (FIG. 1A) and NOXO1 (FIG. 1B) in human tissues. The expression levels of NOX1 and NOXO1 mRNA were measured with real-time RT-PCR. The target gene expression was normalized to expression of β-Actin. The value of mean (N=12) is marked with a horizontal line. *, ** denotes the significance levels between the groups.

FIGS. 2A-2B show NOX1 expression and cell growth in selected stable clones. FIG. 2A: Gene expression was measured by real-time PCR; bars represent the mean of NOX1 expression normalized to β-actin from 3-10 samples of different passages of stable clones (9-17). Error bars represent standard error, * p<0.0001, ** P<0.001 (Student t test). FIG. 2B: Cell growth was measured by doubling time. Five hundred thousand cells were plated for each clone in duplicate, harvested after 72 hrs and counted. The average (Mean±SEM) doubling time was calculated from three independent experiments.

FIG. 3 shows ROS production in stable clones. One million cells were incubated with DCF-DA and number of cells and changes in fluorescence compared to no DCF-DA treated control was determined by flow cytometry. The data is representative of three experiments.

FIG. 4 shows cell cycle analysis of HT-29 stable clones. Cell cycle progression was analyzed in HT-29 human colon cancer cells stably transfected with shRNA against NOX1 (clone 6A) or non-genome specific shRNA (clone SA). HT-29 parental cells are shown as control. Synchronized exponentially growing cells were analyzed by FACS after 0, 24, 48 and 72 h. The numerical data corresponds to the percentage values for the indicated stages of the cell cycle. Data shown is representative of three independent experiments.

FIG. 5 shows HT-29 xenograft in athymic mice. Xenografts were established in 6- to 8-week old male athymic mice (Charles River Laboratories, Frederick, MD). Eight mice in each group were injected subcutaneously with 0.5×10⁶ cells bilaterally. Tumor volumes were calculated from bi-dimensional measurements using the formula 0.5×L×W². Points represent the average tumor volume±SEM of 16 tumors. P values were determined from a two-way analysis of variance.

FIG. 6 shows immunohistochemistry analysis of microvessel formation in HT-29 xenograft. The harvested tumors were fixed in IHC zinc fixative and stained with CD31 antibody. Representative photographs have been shown for H&E-staining and tumor vessel CD31 staining (yellowish-brown) of HT-29, clone SA and 6A xenograft samples. Three investigators counted the numbers of newly formed blood vessels at five fields per section for eight tumors per condition using Leica DM IRB microscope at 20X magnification.

FIG. 7 shows microarray analysis of HT-29 clones and xenografts. The normalized gene expression data was clustered by GeneSpring software. Differences between parental, clone SA and clone 6A are shown in green (down-regulated) and red (up-regulated). Differences in regions of the heat-map between cells and xenografts are most likely the result of the host defense reaction of the mouse tissues against the injected human tumor cells. The lanes represent the mean expression value of at least 3 biological replicates.

FIG. 8 shows the effect of NOX1 inhibition by shRNA in HT-29 cells on cell cycle proteins. Stable clones were selected as described in methods. The protein levels of Cyclin D1 and E, CDK 2, 4 and 6 and p-21/waf and p-27/kip were examined by western blotting analysis in synchronized cells (0-96h). Uniformity of protein loading was confirmed by β-actin. Data shown are representative of at least three independent experiments.

FIG. 9 shows the effect of NOXl inhibition by shRNA in HT-29 cells on selected proteins. Stable clones were selected as described. The protein levels of c-MYB, c-MYC, VEGF-A, VEGFR1 and CXCR-4 were examined by western blot analysis. Uniformity of protein loading was confirmed by either β-actin or β-tubulin. Data shown are representative of at least three independent experiments.

FIG. 10 shows the effect of the NOX1 silencing in epithelial cells.

### DETAILED DESCRIPTION OF THE INVENTION

The present invention relates to compositions and methods for gene-specific inhibition of gene expression by short interfering ribonucleic acid (siRNA) effector molecules. The compositions and methods are particularly useful in modulating gene expression of the NOX1 gene in colon cancer cells.

Seven members of the NADPH oxidase (NOX) family of membrane flavin dehydrogenases have been described. NOX2, formerly designated gp91^{phox}, has been well characterized and is known to produce reactive oxygen species (ROS) as a function of cellular host defense. The biological function of ROS generated by other family members is less understood. Low levels of ROS are thought to play a role in many processes including cell signaling, cell proliferation, inflammatory response, and mitogenesis. NOX1 over-expression in colon cancer cell lines as well as surgical specimens suggests a role in colon cancer.

To confirm a role of NOX1 in colon cancer, the present invention identifies unique target sequences of siRNAs for post-transcriptional silencing of NOX1. As described herein, a U6 promoter/shRNA cassette was cloned into pQBI25-fA1, which expresses the marker GFP (green fluorescent protein). Transient transfection of the constructs into human colon cancer cell lines down-regulated NOX1 expression in sorted cells. Clones of HT-29 cells transfected with the vector that targets base pairs 812-832 of NOX1 (site #6) were selected in G418. Cells expanded from stable clones 6A and 6C demonstrated a 75-80% decrease in NOX1 mRNA expression and a significant decrease in ROS production. Doubling times of clones 6A and 6C increased two to three fold compared to parental cells or HT-29 cells transfected with a non-genome specific shRNA sequence (clone SA) indicating suppressed cell proliferation. HT-29 and clone SA cells completed a cell cycle in 24 hours while clone 6A demonstrated a transient GI/S block, requiring 72 hours to complete a cycle as measured by flow cytometry. Based on the in vitro data, clones 6A and SA and parental HT-29 cells were utilized to establish xenografts in athymic mice to investigate the effect of NOX1 silencing on tumor growth and gene expression in vivo. A significant inhibition of tumor volume was measured in clone 6A tumors (34±4 mm³) compared to clone SA (298±35 mm³) or parental tumors (305±44 mm³) after 27 days (p<0.01). CD31 immunohistochemistry showed a reduction in the number and size of newly formed blood vessels in clone 6A tumors. Microarray analysis and subsequent validation by real time PCR and western blot of stable clones and the corresponding xenograft tissues demonstrated changes in expression of many genes including key regulators of cell proliferation and angiogenesis. Notable examples include the down-regulation of oncogenes c-MYC, c-FOS and c-MYB measured in clone 6A and clone 6A xenografts. Up-regulation of TGFβ1 and CDKN2C (p18), an inhibitor of the CDK4/cyclinD complex, was consistent with the evidence of a transient G1/S arrest observed in clone 6A. CXCR4, a positive regulator of tumor growth and angiogenesis, was significantly down regulated in both clone 6A cells and tumors. HMOX1 and VEGFA, key regulators of angiogenesis, were significantly attenuated in clone 6A tumors supporting the evidence of reduced vessel formation. Our findings demonstrate that silencing of NOX1 inhibited ROS production, cell proliferation and the rate of tumor growth in vivo in part due to antiangiogenic effects downstream of NOX1. The high levels of NOX1 expression in most human colon cancers and the results of these studies confirm NOX1 as a therapeutic target for this disease.

As described below with reference to the specific Examples, this study shows that inhibition of the NOX1 gene with short hairpin RNA decreased the amount of the generated ROS in colon cancer cells leading to the down-regulation of many biological processes, such as cell proliferation, cell cycle regulation, and angiogenesis.

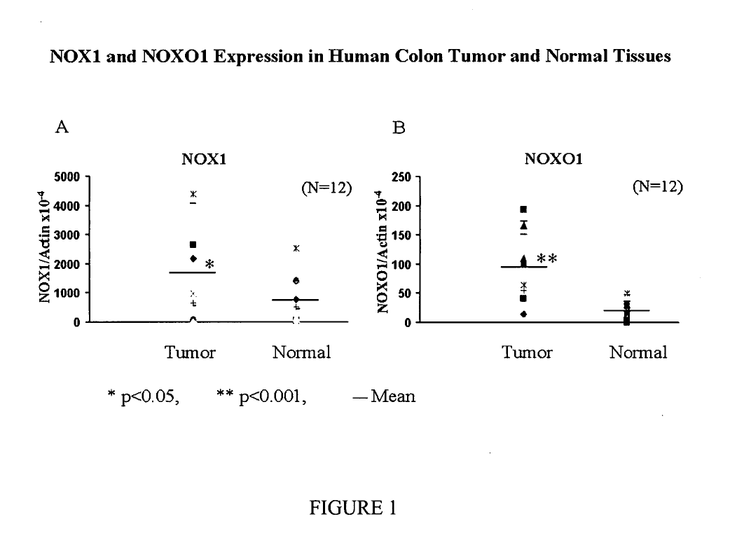

We analyzed the membrane bound p91phox homolog NOX1 catalytic subunit and the accessory proteins NOXO1, NOXA1 and p22phox mRNA expression in surgical samples. Our data shows that the NOX1 transmembrane domain is significantly over-expressed in the tumor samples compared to the corresponding normal tissues (p<0.05, FIG. 1A). In agreement with published data, we detected relatively high NOX1 expression in normal tissue, indicating it is dormant in normal tissue (FIG. 1). Previous studies described NOX1 expression in colon cancer, inflammatory bowel disease (IBD) and in vascular smooth muscle cells (VSMC) (Lassegue et al., 2001; Suh et al., 1999, Banfi et al., 2000; Kikuchi et al., 2000; Fuk-uyama et al., 2005; Shridhar et la., 2004; Sorescu et al., 2002). Recent publications raised questions about the role of NOX1 in colon cancer development. The original theory referred to NOX1 as a mitogenic protein, which plays a significant role in cell growth and tumorgenesis. Other authors question this theory, showing NOX1 is highly expressed in normal colon and inflammatory bowel disease (IBD) samples, not only in tumors, which suggests NOX1 would play a similar “host defense” role in colon as the NOX2 enzyme in phagocytes (Fukuyama et al., 2005; Sorescu et al., 2002). These authors did not study the expression of accessory subunits that are necessary for the formation of the active NOX1 enzyme, however they do not exclude the possibility that the inflammation in the cells can lead to cancerous progression.

We show that the p47phox homolog NOXO1 and the membrane bound p22phox subunits mRNA expression levels were highly significant (p<0.001) in tumors compared to normal tissue in the same sample pairs. This indicates the importance of these subunits in the NOX1 activation and tumorgenesis in colon (FIG. 1B). Our findings are in agreement with recent publications. Cheng et al. (2001) describes 4 splice variants of NOXO1 (α, β, γ, and δ) from different tissues. In colon, NOXO1β is required for activation of NOX1 additionally to NOXA1 (Szanto et al., 2005). The NOXA1 subunit expression did not show significant differences between tumor and adjacent normal tissues. The role of p22phox was studied in the NOX1 and NOX2-dependent ROS generation. Inhibition of p22phox with siRNA, as well as the mutations in the proline-rich region, decreased the reactive oxygen species production, suggesting the importance of the interaction between the membrane bound subunits NOX1 and p22phox, soluble cytosolic subunits NOXO1, NOXA1 and small GTPases Rac1or Rac2 (Cheng and Lambeth, 2005; Kawahara et al., 2005). The p22phox functional association with NOX1 is shown in vascular smooth muscle cells supporting the theory that p22phox acts as a regulatory molecule besides its structure stabilizing role (Li and Engelhardt, 2006).

In conclusion, inhibition of any essential subunits of the activated NOX1 enzyme complex can lead to reduced gene activity, low ROS production and decreased tumor growth.

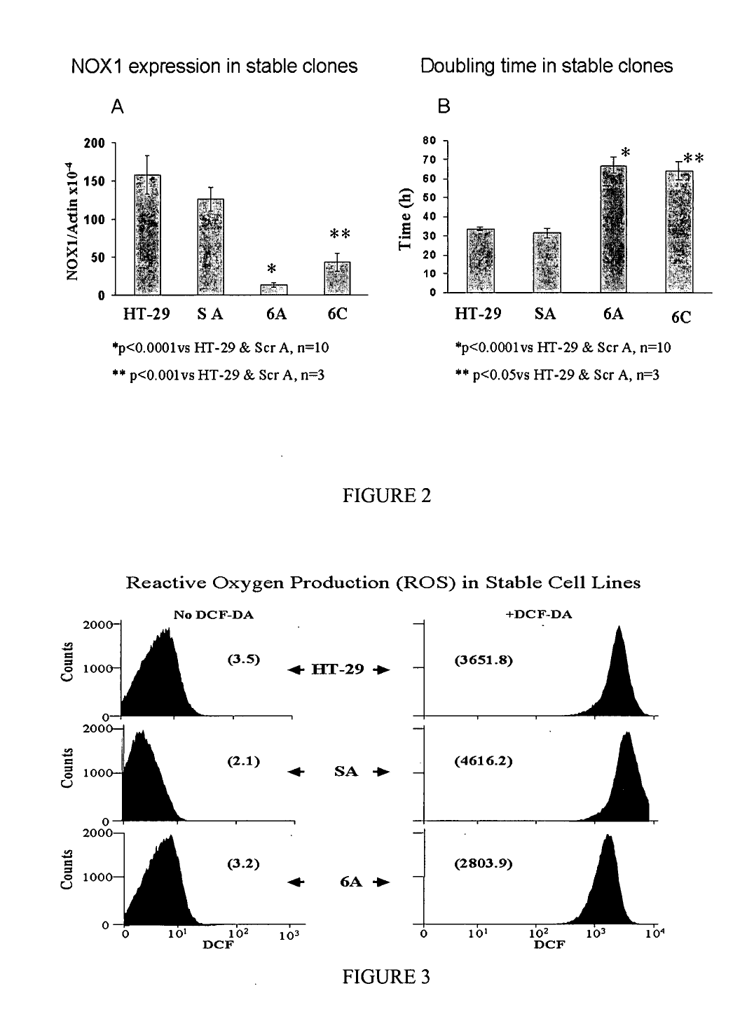

Inhibition of NOX1 catalytic subunit in colon cells with a short-hairpin RNA decreased the cell proliferation in selected stable 6A and 6C clones and attenuated ROS production (FIG. 2B, FIG. 3). A decrease in ROS is the result of silencing and not due to the increased activity of ROS scavengers. We measured no significant difference in the expression of catalase in cells as well as in xenograft tissue (Table 3). Glutathione S-transferase A4 (GSTA4) and GPX3 expression was decreased 3 to 4-fold according to our microarray data (Table 1). These data are in agreement with previously published results that state that NOX1-dependent superoxide production is decreased by inhibition of gene expression with antisense Nox1, as well as removal of ROS with addition of N-acetyl cystein (NAC) or catalase, which leads to growth inhibition in cells (Arnold et al., 2001; Suh et al., 1999).

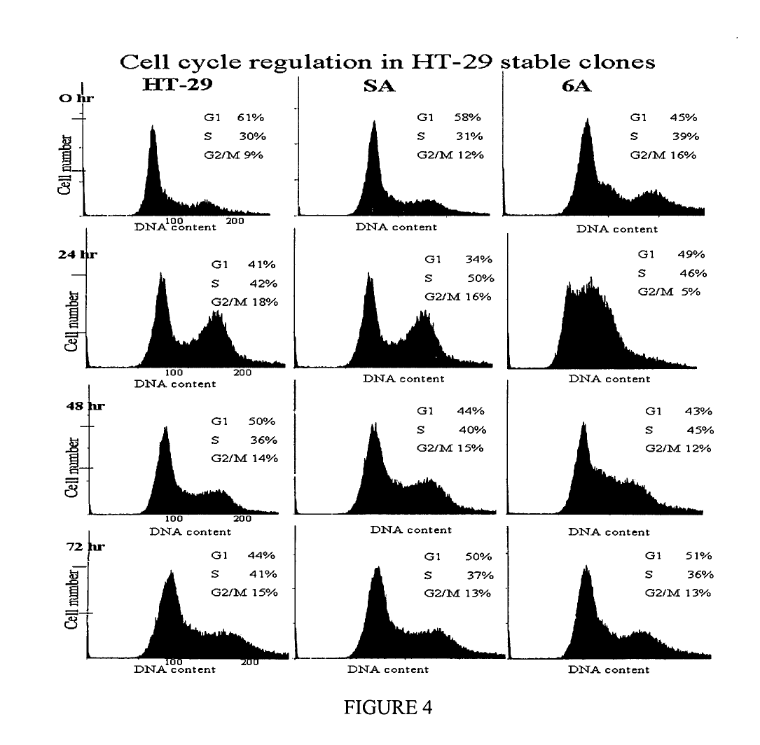

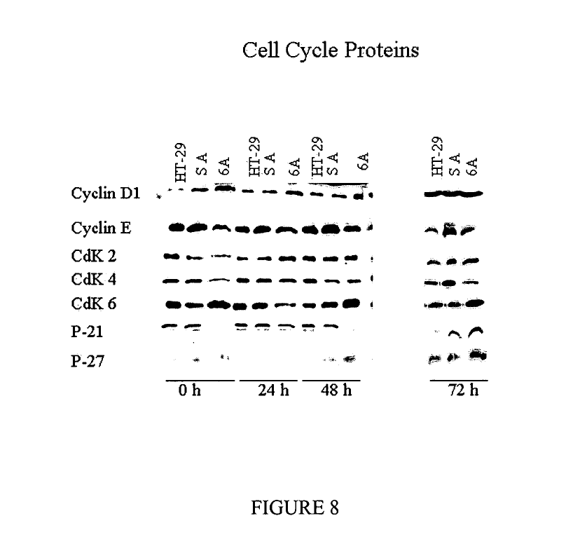

ROS removal leads to changes in cell cycle regulation, which has been shown by measuring the cell proliferation with doubling time (FIG. 2B). Flow cytometry, micro-array, real-time PCR and western blot results suggest the existence of a transient GC/S block as shown in FIG. 4, FIG. 8 and Table 3. Oncogenes such as c-MYC, c-MYB and c-FOS, which were down regulated by silencing NOX1, play regulatory roles in the transcription of cell cycle checkpoint genes. It was implicated earlier that intracellular redox-state regulates the Go/G1 phase signaling and controls the progression to S phase via activation of G1 regulatory proteins, especially D-type cyclins, CDK4/6 and their inhibitors, which are members of the Cip/Kip and INK4 families (Hanna et al., 2004). In our study, clone 6A showed a decrease in CDK4 protein by western-blot analysis. We also showed a significant up-regulation of the INK4 inhibitor, p18 (CDKN2C) by real-time PCR (Table 3.). Furthermore, we demonstrated that increased expression of TGF-β induces p18 transcription and GC/S arrest (Table 3). Zhang et al. (1999) proposed that TGF-β arrests the cells in two distinct stages of the cell cycle: in G1 by induction of the Rb-E2F repressor and in G2/M as a result of decreased CDK2 activity (Menon et al., 2003). The Cip/Kip family members (Cip1 or p21 and Kip2 or p57) are attenuated in xenografts which may be sufficient to overcome the G1 block so the cell cycle can progress (Zhang et al., 1999). C-MYB was identified as part of the cyclin-dependent kinase containing complex and its activity is directly regulated by cyclinD1/CDK4 or CDK6 in hematopoietic cells (Sherr et al., 1999). Recent studies show the IFI16, interferon-γ-inducible protein 16, has a cell cycle regulatory role in the expression of E2F1, cyclin DI and Cip1 (p2l). In addition, IF116 and PIAS3 (STAT3 inhibitor) are affecting growth arrest through the JAK/STAT3 signaling pathway in medullary thyroid carcinoma cells (Lei et al., 2005; Kim et al., 2005). Kawahara et al. (2005) have found interferon-y-responsive elements in the promoter region of the NOX1 gene in human large intestinal epithelial cells. JAK kinase inhibitor blocked NOX1 mRNA expression and superoxide production, suggesting the crucial role of STAT1 in the interferon-γ-stimulated transcription of NOX1 gene (Hodge et al., 2005). Whether the observed IFI16 overexpression in our xenograft samples is related to NOX1 silencing directly or indirectly, has to be determined (Table 2).

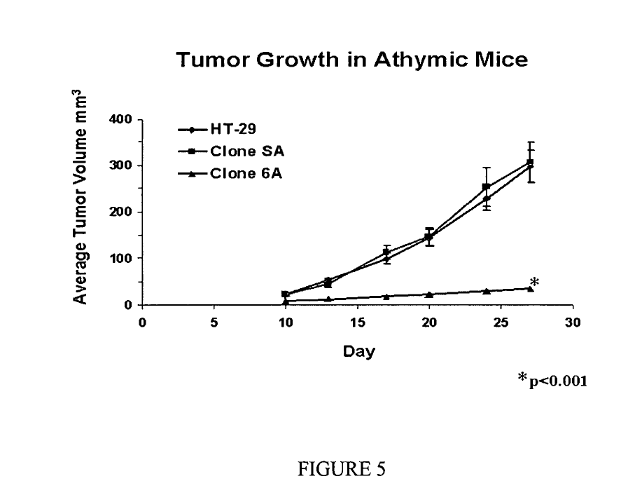

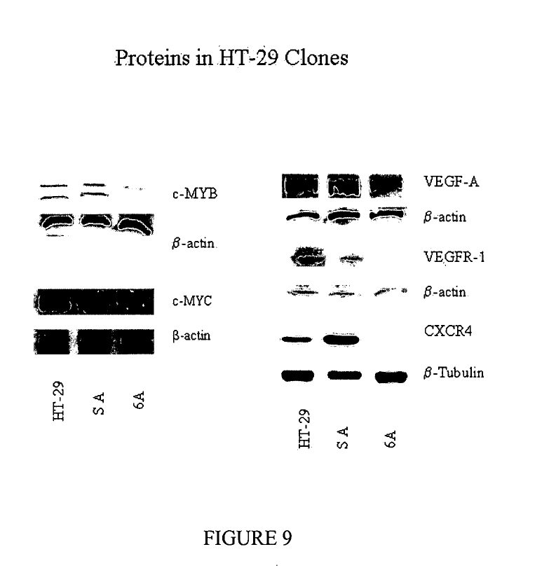

As previously reported, the ROS generated by the active NOX1 enzyme complex triggers the angiogenic switch (Arbiser et al., 2002). We have shown in this study that tumor growth is significantly depressed in the clone 6A xenograft compared to SA and HT-29 (FIG. 5). The number of the newly formed vessels decreased, their lumen shrank as the CD31 (PECAM1) staining showed and expression of genes, which are important for angiogenesis, were down-regulated (Table 3, FIG. 9.). It is still poorly understood how the ROS level influences angiogenetic pathways and how the cell cycle deregulation leads to tumor formation. The major stimulus for the pro-angiogenic switch is the reduced availability of oxygen, hypoxia and the increased expression of oncogenes (Kuwano et al., 2006). VEGF-A, which is under regulation of hypoxia-inducible factor (HIF1) in hypoxic conditions, and its receptor, VEFR1, showed decreased expression (Table 3, FIG. 9) (Kniew-Bamforth et al., 2004). VEGF-A, a key regulator of angiogenic processes, is controlled by c-MYC, stimulates the circulation of angiogenic precursors and recruits them to the newly formed vessels (Semenza, 2003).

In our study we were not able to detect the changes in mRNA expression of HIF-1α, but heme oxygenase-1 (HMOX1), which is down-stream from HIF1-α and CA9, a hypoxia marker, is closely associated with HIF-1α, were decreased (Table 3, Table 1)(Mezquita et al., 2005). This is in agreement with findings of Goyal et al. (2004), who suggest that a hypoxic up-regulation of NOX1 expression augments ROS production, which may in turn activate the HIF1α-dependent pathways and increase transcription of HIF1α-dependent genes in lung cancer cell lines (Berberat et al., 2005).

In a recent paper, Grunewald et al. (2006) describe a model how VEGF-A and the CXCR4 chemokine receptor play a role in formation of new vessels. CXCR4 is regulated by hypoxia and siRNA inhibition of mRNA expression decreased the invasion of breast cancer cells in vitro (Schoppa et al., 2003; Grunewald et al., 2006; Saccani et al., 2000). In colon cancer, blocking the SDF-1/CXCR4 axis by anti-CXCR4 antibody attenuates the tumor growth in colon cancer xenografts (Chen et al., 2003; Ottaiano et al., 2005). These data are in agreement with our measurements, suggesting that NOX1 silencing has an important effect through regulation of the ROS level on the CXCR4 receptor activity (Table 3). We detected the highly significant inhibition of another chemokine, CCL15, which stimulates endothelial cell migration, stimulates new vessel formation and mediates angiogenesis. This suggests, that chemokines and chemokine receptors play an important role in the ROS-mediated pro-angiogenic biological pathways (Table 3) (Hwang et al., 2004).

Adrenomedullin (ADM) is a novel vasodilating protein, which has angiogenic properties in human tumors by enhancing the VEGFA-induced capillary formation and Akt activity in sarcomal80 xenograft in mice (limuro et al., 2004). Its suppressed expression is an indication of the inhibition of angiogenesis in our NOX1 shRNA knockout xenograft samples (Table 3).

The dividing tumor cells require enormous amounts of nutrients, especially glucose, to meet their high metabolic requirements. Oncogenic transformation or activation of HIF-dependent pathways, even in normoxia, can stimulate the key regulatory enzymes of glycolysis (HK2, SI, SHMT2 and IDH2) or mobilize the alternative energetic pathways by degradation of cellular proteins and aminoacids (Guleng et al., 2005). We measured reduced expression of many genes that are affected by the reduction of ROS and involved in various metabolic pathways, including aldo-keto reductase family members (AKR1B10 and AKR1C1), as well as iron transport and metabolism family members (TFRC, HMOX1 and HEPH). We observed significant reduction in the expression levels of the aminoacid, glucose, Cu2+, Ca2+, and potassium transporting proteins by microarray (Table 3). The role of these genes has to be determined.

There are contradictory data about the role of the AXL tyrosine kinase receptor and its ligand GAS6 in tumors. We determined significant overexpression in cells, as well as in xenograft tissue. They are over-expressed in inflammatory kidney diseases and tumor growth was suppressed in MDA-MB-231 xenograft transfected with shAXL vectors. This implies that AXL plays a key role in driving the growth of human tumor cells in vivo (North et al., 2005; Fiebeler et al., 2004). However, Gallicchio et al. (2005) reported that AXL stimulation by GAS6 resulted in inhibition of VEGFR2 ligand-dependent activation and the inhibition of the angiogenic process. GAS6 inhibits the chemotaxis of endothelial cells stimulated by VEGFA and vascularization (Holland et al., 2005). We observed up-regulation of the AXL/GAS6 pathway in vitro and in vivo, which raises a question about the role of these genes. The increased expression may be anti-angiogenic or pro-angiogenic (Table 3) We cannot rule out either their VEGFR2 inhibitory role or their possible crosstalk with the integrins in stimulating the growth, migration and survival of tumor cells.

As we mentioned, tumor growth in the clone 6A xenograft was not inhibited completely. We observed the activation of a group of pro-angiogenic genes, which are involved in extracellular matrix degradation, tumor cell migration and proliferation, including integrins (ITGB5), metalloproteinases (MMP14, ADAM28) and urokinase (PLAU) (FIG. 5, Table 2). These data suggest that with the inhibition of the ROS production by NOX1 we were not able to suppress all of the pathways leading to angiogenesis. Inhibition of one gene is insufficient to eliminate the tumor growth. A combination of inhibitors may be required for successful anti cancer therapies.

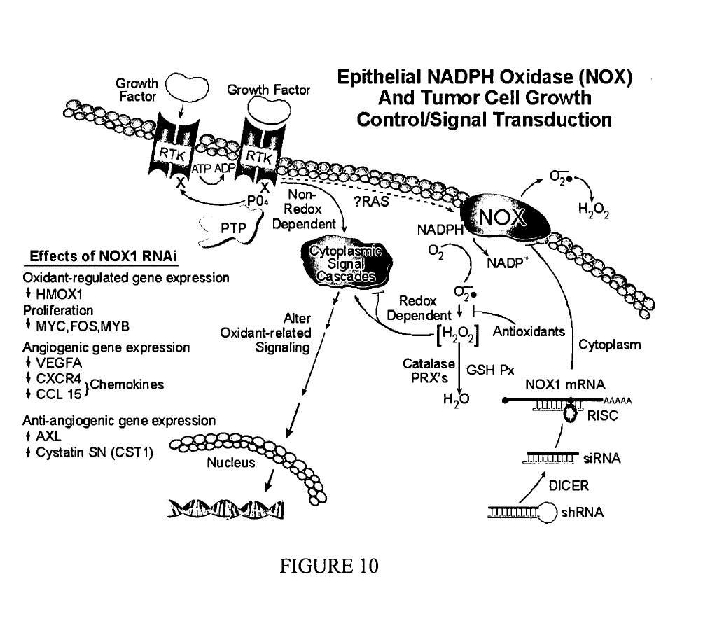

Taken together as FIG. 10. shows shRNA silencing of NOX1 gene reduces the amount of the generated ROS. This change affects many signaling pathways, which have genes sensitive to oxido-reductive conditions. These involve tyrosine kinase receptors (AXL), chemokine receptors (CXCR4), growth factor receptors (FGFR3, INSR), secreted signaling molecules (VEGF-A, TGFβ1, c-MYC, c-FOS, c-MYB) and important metabolic genes (glycolysis, aminoacid transport, iron transport). We have shown evidence that ROS level regulates the cell cycle and angiogenesis in NOX1 expressing colon cancer. These studies strongly support that NOX1 is a therapeutic target for this disease.

The siRNA molecule may have different forms, including a single strand, a paired double strand (dsRNA) or a hairpin (shRNA) and can be produced, for example, either sythetically or by expression in cells. In one embodiment, DNA sequences for encoding the sense and antisense strands of the siRNA molecule to be expressed directly in mammalian cells can be produced by methods known in the art, including but not limited to, methods described in U.S. published application Nos. 2004/0171118 A1, 2005/0244858 A1 and 2005/0277610 A1, each incorporated herein by reference.

In one aspect of the invention, DNA sequences encoding a sense strand and an antisense strand of a siRNA specific for a target sequence of a gene are introduced into mammalian cells for expression. To target more than one sequence in the gene (such as different promoter region sequences and/or coding region sequences), separate siRNA-encoding DNA sequences specific to each targeted gene sequence can be introduced simultaneously into the cell. In accordance with another embodiment, mammalian cells may be exposed to multiple siRNAs that target multiple sequences in the gene.

The siRNA molecules generally contain about 19 to about 30 base pairs, and preferably are designed to cause methylation of the targeted gene sequence. In one embodiment, the siRNA molecules contain about 19-23 base pairs, and preferably about 21 base pairs. In another embodiment, the siRNA molecules contain about 24-28 base pairs, and preferably about 26 base pairs. In a further embodiment, the dsRNA has an asymmetric structure, with the sense strand having a 25-base pair length, and the antisense strand having a 27-base pair length with a 2 base 3′-overhang. In another embodiment, this dsRNA having an asymmetric structure further contains 2 deoxynucleotides at the 3′ end of the sense strand in place of two of the ribonucleotides.Individual siRNA molecules also may be in the form of single strands, as well as paired double strands (“sense” and “antisense”) and may include secondary structure such as a hairpin loop. Individual siRNA molecules could also be delivered as precursor molecules, which are subsequently altered to give rise to active molecules. Examples of siRNA molecules in the form of single strands include a single stranded anti-sense siRNA against a non-transcribed region of a DNA sequence (e.g. a promoter region).

The sense and antisense strands anneal under biological conditions, such as the conditions found in the cytoplasm of a cell. In addition, a region of one of the sequences, particularly of the antisense strand, of the dsRNA has a sequence length of at least 19 nucleotides, wherein these nucleotides are adjacent to the 3′ end of antisense strand and are sufficiently complementary to a nucleotide sequence of the RNA produced from the target gene.

The precursor RNAi molecule, may also have one or more of the following additional properties: (a) the antisense strand has a right shift from the typical 21mer and (b) the strands may not be completely complementary, i.e., the strands may contain simple mismatch pairings. A “typical” 21mer siRNA is designed using conventional techniques, such as described above. This 21mer is then used to design a right shift to include 1-7 additional nucleotides on the 5′ end of the 21mer. The sequence of these additional nucleotides may have any sequence. Although the added ribonucleotides may be complementary to the target gene sequence, full complementarity between the target sequence and the antisense siRNA is not required. That is, the resultant antisense siRNA is sufficiently complementary with the target sequence. The first and second oligonucleotides are not required to be completely complementary. They only need to be substantially complementary to anneal under biological conditions and to provide a substrate for Dicer that produces an siRNA sufficiently complementary to the target sequence. In one embodiment, the dsRNA has an asymmetric structure, with the antisense strand having a 25-base pair length, and the sense strand having a 27-base pair length with a 2 base 3′-overhang. In another embodiment, this dsRNA having an asymmetric structure further contains 2 deoxynucleotides at the 3 ′ end of the antisense strand.

Suitable dsRNA compositions that contain two separate oligonucleotides can be linked by a third structure. The third structure will not block Dicer activity on the dsRNA and will not interfere with the directed destruction of the RNA transcribed from the target gene. In one embodiment, the third structure may be a chemical linking group. Many suitable chemical linking groups are known in the art and can be used. Alternatively, the third structure may be an oligonucleotide that links the two oligonucleotides of the dsRNA is a manner such that a hairpin structure is produced upon annealing of the two oligonucleotides making up the dsRNA composition. The hairpin structure will not block Dicer activity on the dsRNA and will not interfere with the directed destruction of the RNA transcribed from the target gene.

The sense and antisense sequences may be attached by a loop sequence. The loop sequence may comprise any sequence or length that allows expression of a functional siRNA expression cassette in accordance with the invention. In a preferred embodiment, the loop sequence contains higher amounts of uridines and guanines than other nucleotide bases. The preferred length of the loop sequence is about 4 to about 9 nucleotide bases, and most preferably about 8 or 9 nucleotide bases.

In another embodiment of the present invention, the dsRNA, i.e., the precursor RNAi molecule, has several properties which enhances its processing by Dicer. According to this embodiment, the dsRNA has a length sufficient such that it is processed by Dicer to produce an siRNA and at least one of the following properties: (i) the dsRNA is asymmetric, e.g., has a 3′ overhang on the sense strand and (ii) the dsRNA has a modified 3′ end on the antisense strand to direct orientation of Dicer binding and processing of the dsRNA to an active siRNA. According to this embodiment, the longest strand in the dsRNA comprises 24-30 nucleotides. In one embodiment, the sense strand comprises 24-30 nucleotides and the antisense strand comprises 22-28 nucleotides. Thus, the resulting dsRNA has an overhang on the 3′ end of the sense strand. The overhang is 1-3 nucleotides, such as 2 nucleotides. The antisense strand may also have a 5′ phosphate.

Modifications can be included in the dsRNA, i.e., the precursor RNAi molecule, so long as the modification does not prevent the dsRNA composition from serving as a substrate for Dicer. In one embodiment, one or more modifications are made that enhance Dicer processing of the dsRNA. In a second embodiment, one or more modifications are made that result in more effective RNAi generation. In a third embodiment, one or more modifications are made that support a greater RNAi effect. In a fourth embodiment, one or more modifications are made that result in greater potency per each dsRNA molecule to be delivered to the cell. Modifications can be incorporated in the 3′-terminal region, the 5′-terminal region, in both the 3′-terminal and 5′-terminal region or in some instances in various positions within the sequence. With the restrictions noted above in mind any number and combination of modifications can be incorporated into the dsRNA. Where multiple modifications are present, they may be the same or different. Modifications to bases, sugar moieties, the phosphate backbone, and their combinations are contemplated. Either 5′-terminus can be phosphorylated.

In another embodiment, the antisense strand is modified for Dicer processing by suitable modifiers located at the 3′ end of the antisense strand, i.e., the dsRNA is designed to direct orientation of Dicer binding and processing. Suitable modifiers include nucleotides such as deoxyribonucleotides, dideoxyribonucleotides, acyclonucleotides and the like and sterically hindered molecules, such as fluorescent molecules and the like. Acyclonucleotides substitute a 2-hydroxyethoxymethyl group for the 2′-deoxyribofuranosyl sugar normally present in dNMPs. Other nucleotide modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidine (d4T) and the monophosphate nucleotides of 3′-azido-3′-deoxythymidine (AZT), 2′,3′-dideoxy-3′-thiacytidine (3TC) and 2′,3′-didehydro-2′,3′-dideoxythymidine (d4T). In one embodiment, deoxynucleotides are used as the modifiers. When nucleotide modifiers are utilized, 1-3 nucleotide modifiers, or 2 nucleotide modifiers are substituted for the ribonucleotides on the 3′ end of the antisense strand. When sterically hindered molecules are utilized, they are attached to the ribonucleotide at the 3′ end of the antisense strand. Thus, the length of the strand does not change with the incorporation of the modifiers. In another embodiment, the invention contemplates substituting two DNA bases in the dsRNA to direct the orientation of Dicer processing. In a further invention, two terminal DNA bases are located on the 3′ end of the antisense strand in place of two ribonucleotides forming a blunt end of the duplex on the 5′ end of the sense strand and the 3′ end of the antisense strand, and a two-nucleotide RNA overhang is located on the 3′-end of the sense strand. This is an asymmetric composition with DNA on the blunt end and RNA bases on the overhanging end.

Examples of modifications contemplated for the phosphate backbone include phosphonates, including methylphosphonate, phosphorothioate, and phosphotriester modifications such as alkylphosphotriesters, and the like. Examples of modifications contemplated for the sugar moiety include 2′-alkyl pyrimidine, such as 2′-O-methyl, 2′-fluoro, amino, and deoxy modifications and the like (see, e.g., Amarzguioui et al., 2003). Examples of modifications contemplated for the base groups include abasic sugars, 2-O-alkyl modified pyrimidines, 4-thiouracil, 5-bromouracil, 5-iodouracil, and 5-(3-aminoallyl)-uracil and the like. Locked nucleic acids, or LNA's, could also be incorporated. Many other modifications are known and can be used so long as the above criteria are satisfied. Examples of modifications are also disclosed in U.S. Pat. Nos. 5,684,143, 5,858,988 and 6,291,438 and in U.S. published patent application No. 2004/0203145 A1, each incorporated herein by reference. Other modifications are disclosed in Herdewijn (2000), Eckstein (2000), Rusckowski et al. (2000), Stein et al. (2001) and Vorobjev et al. (2001).

Additionally, the siRNA structure can be optimized to ensure that the oligonucleotide segment generated from Dicer's cleavage will be the portion of the oligonucleotide that is most effective in inhibiting gene expression. For example, in one embodiment of the invention a 27-bp oligonucleotide of the dsRNA structure is synthesized wherein the anticipated 21 to 22-bp segment that will inhibit gene expression is located on the 3′-end of the antisense strand. The remaining bases located on the 5′-end of the antisense strand will be cleaved by Dicer and will be discarded. This cleaved portion can be homologous (i.e., based on the sequence of the target sequence) or non-homologous and added to extend the nucleic acid strand.

RNA may be produced enzymatically or by partial/total organic synthesis, and modified ribonucleotides can be introduced by in vitro enzymatic or organic synthesis. In one embodiment, each strand is prepared chemically. Methods of synthesizing RNA molecules are known in the art, in particular, the chemical synthesis methods as described in Verma and Eckstein (1998) or as described herein.

In another aspect, the present invention provides for a pharmaceutical composition comprising the siRNA of the present invention. The siRNA sample can be suitably formulated and introduced into the environment of the cell by any means that allows for a sufficient portion of the sample to enter the cell to induce gene silencing, if it is to occur. Many formulations for dsRNA are known in the art and can be used so long as siRNA gains entry to the target cells so that it can act. See, e.g., U.S. published patent application Nos. 2004/0203145 A1 and 2005/0054598 A1, each incorporated herein by reference. For example, siRNA can be formulated in buffer solutions such as phosphate buffered saline solutions, liposomes, micellar structures, and capsids. Formulations of siRNA with cationic lipids can be used to facilitate transfection of the dsRNA into cells. For example, cationic lipids, such as lipofectin (U.S. Pat. No. 5,705,188, incorporated herein by reference), cationic glycerol derivatives, and polycationic molecules, such as polylysine (published PCT International Application WO 97/30731, incorporated herein by reference), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

It can be appreciated that the method of introducing siRNA into the environment of the cell will depend on the type of cell and the make up of its environment. For example, when the cells are found within a liquid, one preferable formulation is with a lipid formulation such as in lipofectamine and the siRNA can be added directly to the liquid environment of the cells. Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate siRNA in a buffer or saline solution and directly inject the formulated dsRNA into cells, as in studies with oocytes. The direct injection of dsRNA duplexes may also be done. For suitable methods of introducing siRNA see U.S. published patent application No. 2004/0203145 A1, incorporated herein by reference.

Suitable amounts of siRNA must be introduced and these amounts can be empirically determined using standard methods. Typically, effective concentrations of individual siRNA species in the environment of a cell will be about 50 nanomolar or less 10 nanomolar or less, or compositions in which concentrations of about 1 nanomolar or less can be used. In other embodiment, methods utilize a concentration of about 200 picomolar or less and even a concentration of about 50 picomolar or less can be used in many circumstances.

The method can be carried out by addition of the siRNA compositions to any extracellular matrix in which cells can live provided that the siRNA composition is formulated so that a sufficient amount of the siRNA can enter the cell to exert its effect. For example, the method is amenable for use with cells present in a liquid such as a liquid culture or cell growth media, in tissue explants, or in whole organisms, including animals, such as mammals and especially humans.

Expression of a target gene can be determined by any suitable method now known in the art or that is later developed. It can be appreciated that the method used to measure the expression of a target gene will depend upon the nature of the target gene. For example, when the target gene encodes a protein the term “expression” can refer to a protein or transcript derived from the gene. In such instances the expression of a target gene can be determined by measuring the amount of mRNA corresponding to the target gene or by measuring the amount of that protein. Protein can be measured in protein assays such as by staining or immunoblotting or, if the protein catalyzes a reaction that can be measured, by measuring reaction rates. All such methods are known in the art and can be used. Where the gene product is an RNA species expression can be measured by determining the amount of RNA corresponding to the gene product. The measurements can be made on cells, cell extracts, tissues, tissue extracts or any other suitable source material.

The determination of whether the expression of a target gene has been reduced can be by any suitable method that can reliably detect changes in gene expression. Typically, the determination is made by introducing into the environment of a cell undigested siRNA such that at least a portion of that siRNA enters the cytoplasm and then measuring the expression of the target gene. The same measurement is made on identical untreated cells and the results obtained from each measurement are compared.

The siRNA can be formulated as a pharmaceutical composition which comprises a pharmacologically effective amount of a siRNA and pharmaceutically acceptable carrier. A pharmacologically or therapeutically effective amount refers to that amount of a siRNA effective to produce the intended pharmacological, therapeutic or preventive result. The phrases “pharmacologically effective amount” and “therapeutically effective amount” or simply “effective amount” refer to that amount of an RNA effective to produce the intended pharmacological, therapeutic or preventive result. For example, if a given clinical treatment is considered effective when there is at least a 20% reduction in a measurable parameter associated with a disease or disorder, a therapeutically effective amount of a drug for the treatment of that disease or disorder is the amount necessary to effect at least a 20% reduction in that parameter.

The phrase “pharmaceutically acceptable carrier” refers to a carrier for the administration of a therapeutic agent. Exemplary carriers include saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. The pharmaceutically acceptable carrier of the disclosed dsRNA composition may be micellar structures, such as a liposomes, capsids, capsoids, polymeric nanocapsules, or polymeric microcapsules.

Polymeric nanocapsules or microcapsules facilitate transport and release of the encapsulated or bound dsRNA into the cell. They include polymeric and monomeric materials, especially including polybutylcyanoacrylate. A summary of materials and fabrication methods has been published (see Kreuter, 1991). The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Suitably formulated pharmaceutical compositions of this invention can be administered by any means known in the art such as by parenteral routes, including intravenous, intramuscular, intraperitoneal, subcutaneous, transdermal, airway (aerosol), rectal, vaginal and topical (including buccal and sublingual) administration. In some embodiments, the pharmaceutical compositions are administered by intravenous or intraparenteral infusion or injection.

In general a suitable dosage unit of siRNA will be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. Pharmaceutical composition comprising the siRNA can be administered once daily. However, the therapeutic agent may also be dosed in dosage units containing two, three, four, five, six or more sub-doses administered at appropriate intervals throughout the day. In that case, the siRNA contained in each sub-dose must be correspondingly smaller in order to achieve the total daily dosage unit. The dosage unit can also be compounded for a single dose over several days, e.g., using a conventional sustained release formulation which provides sustained and consistent release of the siRNA over a several day period. Sustained release formulations are well known in the art. In this embodiment, the dosage unit contains a corresponding multiple of the daily dose. Regardless of the formulation, the pharmaceutical composition must contain siRNA in a quantity sufficient to inhibit expression of the target gene in the animal or human being treated. The composition can be compounded in such a way that the sum of the multiple units of siRNA together contain a sufficient dose.

Data can be obtained from cell culture assays and animal studies to formulate a suitable dosage range for humans. The dosage of compositions of the invention lies within a range of circulating concentrations that include the ED₅₀ (as determined by known methods) with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound that includes the IC₅₀ (i.e., the concentration of the test compound which achieves a half-maximal inhibition of symptoms) as determined in cell culture. Such information can be used to more accurately determine useful doses in humans. Levels of dsRNA in plasma may be measured by standard methods, for example, by high performance liquid chromatography.

In a further aspect, the present invention relates to a method for treating a subject having colon cancer or at risk of developing colon cancer associated with the high level of expression of NOX1. The method comprises introducing the siRNA into appropriate cancer tissue. The term “introducing” encompasses a variety of methods of introducing DNA into a cell, either in vitro or in vivo. Such methods include transformation, transduction, transfection, and infection. Vectors are useful and preferred agents for introducing DNA encoding the siRNA molecules into cells. The introducing may be accomplished using at least one vector. Possible vectors include plasmid vectors and viral vectors. Viral vectors include retroviral vectors, lentiviral vectors, or other vectors such as adenoviral vectors or adeno-associated vectors. In one embodiment, the DNA sequences are included in separate vectors, while in another embodiment, the DNA sequences are included in the same vector. The DNA sequences may be inserted into the same vector as a multiple cassettes unit. Alternate delivery of siRNA molecules or DNA encoding siRNA molecules into cells or tissues may also be used in the present invention, including liposomes, chemical solvents, electroporation, viral vectors, as well as other delivery systems known in the art.

Suitable promoters include those promoters that promote expression of the interfering RNA molecules once operatively associated or linked with sequences encoding the RNA molecules. Such promoters include cellular promoters and viral promoters, as known in the art. In one embodiment, the promoter is an RNA Pol III promoter, which preferably is located immediately upstream of the DNA sequences encoding the interfering RNA molecule. Various viral promoters may be used, including, but not limited to, the viral LTR, as well as adenovirus, SV40, and CMV promoters, as known in the art.

In one embodiment, the invention uses a mammalian U6 RNA Pol III promoter, and more preferably the human U6snRNA Pol III promoter, which has been used previously for expression of short, defined ribozyme transcripts in human cells (Bertrand et al., 1997; Good et al., 1997). The U6 Pol III promoter and its simple termination sequence (four to six uridines) were found to express siRNAs in cells. Appropriately selected interfering RNA or siRNA encoding sequences can be inserted into a transcriptional cassette, providing an optimal system for testing endogenous expression and function of the RNA molecules.

In a further aspect, the invention provides a method for methylating NOX1 in a mammalian cell comprising introducing into the cell DNA sequences encoding a sense strand and an antisense strand of an siRNA, which is specific for a target sequence in the NOX1 gene of interest, preferably under conditions permitting expression of the siRNA in the cell, and wherein the siRNA directs methylation of said gene of interest. In an embodiment, methylation is directed to a sequence in the promoter region of the gene. Alternately, methylation is directed to a sequence in the coding region. Target sequences can be any sequence in a gene that has the potential for methylation. In a preferred embodiment, the target sequences contain CpG islands. The directed methylation can lead to inactivation of the gene.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

### EXAMPLES

The present invention is described by reference to the following Examples, which are offered by way of illustration and are not intended to limit the invention in any manner. Standard techniques well known in the art or the techniques specifically described below were utilized.

### Example 1

### Methods

RNA isolation and reverse transcription: RNA from cell lines was isolated with RNeasy Mini Kit (Qiagen, Valencia, Calif. US). RNA was isolated from tissues after disruption with a Polytron homogenizer using RNA-zol B (TEL-TEST Inc. Friendswood, Tex.) according to the manufacturer's instructions. Genomic DNA contamination was removed with DNase I treatment (Ambion, Austin, Tex.). The samples were purified on mini columns of RNeasy Mini Kit. Integrity of the RNA was tested on 1% agarose gel (SeaKem, FMC, Rockland, Me.) or on Agilent Bioanalyzer 2100 (Agilent Inc. Palo Alto, Calif.). The RNA concentration and A₂₆₀/A₂₈₀ ratio was measured a UV spectrophotometer. cDNA was prepared from 1-10 μg RNA using MMLV reverse transcriptase enzyme and random hexamers as primers according to the manufacturer instructions (Invitrogen, Carlsbad, Calif.). cDNA was stored at −20° C. until used.

Real-time PCR: Real-time PCR was carried out to measure the level of gene expression. One μL cDNA from each sample was used per reaction in final volume of 20 μL as described earlier (Juhasz et al., 2003). The primers and probes were designed according to the Applied Biosystems guidelines (Primer Express software, Applied Biosystems, Foster City, Calif.) for the following genes: NADPH oxidase1 (NOX1), adrenomedullin (ADM), AXL receptor tyrosine kinase (AXL), catalase (CAT), chemokine (C—C motif) ligand 15 (CCL15), cyclin-dependent kinase inhibitor 2C or p18 (CDKN2C), c-FOS, c-MYB, c-MYC, cystatin SN (CST1), chemokine (C—X—C motif) receptor 4 (CXCR4), fibroblast growth factor receptor 3 (FGFR3), growth arrest-specific 6 (GAS6), hypoxia-inducible factor 1, alpha subunit (HIF1α), heme oxygenase (decycling) 1 (HMOX1), transforming growth factor, beta 1 (TGFB1) and vascular endothelial growth factor A (VEGF-A).

Genomic DNA amplification was excluded by designing the primers around the exon-intron splicing sites. The PCR amplification was performed on a 384 well plate using the default cycling conditions (Applied Biosystems, Foster City, Calif.). For the absolute calibration curve of the target genes and the housekeeping gene (18S or β-Actin), serial dilutions of the plasmids (10⁷ to 1 copy range) containing the gene insert were used. NOX1 plasmids were kindly provided by Drs B.Banfi and K-H. Krause (Geneva, Switzerland) and Dr H. Kikuchi (Sendai, Japan). Relative gene expression was determined as the ratio of the gene of interest to the internal reference gene expression based on the standard curves.

shRNA mediated silencing of NOX1 gene: siRNA's were designed against NOX1 (GenBank Accession No: AF166327, GI: 6138993; incorporated herein by reference). After comparing with the human genome database (BLAST), 4 unique sequences were found, which are as follows:

(SEQ ID NO: 1)

| | |
|---|---|
| 1, target sequence | GAGAAGGCCGACAAATACTAC; |
| (170 bp-191 bp): | |

(SEQ ID NO: 2)

| | |
|---|---|
| 2-target sequence | GCTCATTTTGCAGCCGCACAC; |

(318 bp-339 bp):

(SEQ ID NO: 3)

| | |
|---|---|
| 6, target sequence | GAGATGTGGGATGATCGTGAC; |
| (812 bp-832 bp): | |

(SEQ ID NO: 4)

| | |
|---|---|
| 8, target sequence | GGCTTTCGAACAACAATATTC |
| (1171 bp-1191 bp): | |
| and | |

(SEQ ID NO: 5)

| | |
|---|---|
| Scrambled sequence | GTCGTACCGTACGTAAGACAC. |
| (not represented in the hu- | |
| man genome): | |

We used a two-step PCR approach to make human U6 promoter+siRNA constructs, as previously described by J. Rossi's laboratory (Lee et al., 2002; Castanotto et al., 2002). The amplified u6+si RNA cassettes were cloned into the TA cloning site of pCR2.1 (Invitrogen Corp., Carlsbad, Calif.), then subdloned between Kpn I and Xba I restriction sites of pSP73 (Promega Corp., Madison, Wis.). Finally, the expression cassette was subcloned between the Bg1 II and Xho I restriction sites of pQBI25-fA1 GFP (Green Fluorescence Protein) expression vector (Qbiogene, Inc.Irvine, Calif.). TOP10F′ competent cells were transformed, and minipreps were sequenced on an ABI 3730 DNA analyzer (Applied Biosystems, Foster City, Calif.) using a hot start and dGTP chemistry at the City of Hope DNA sequencing core facility.

shRNA Constructs Used in NOX1 Silencing Study: All of the constructs consist of a U6+1 promoter sequence, sense sequence of the target, loop sequence, antisense sequence of the target and a poly T terminator sequence. The constructs had the following structures.

Control Construct Sequence: U6+1 promoter plus a non-genome specific shRNA sequence:

(SEQ ID NO: 6)

| | |
|---|---|
| | U6 promoter |
| 1 | GGATCCAAGG TCGGGCAGGA AGAGGGCCTA TTTCCCATGA |
| | TTCCTTCATA |
| 51 | TTTGCATATA CGATACAAGG CTGTTAGAGA GATAATTAGA |
| | ATTAATTTGA |
| 101 | CTGTAAACAC AAAGATATTA GTACAAAATA CGTGACGTAG |
| | AAAGTAATAA |
| 151 | TTTCTTGGGT AGTTTGCAGT TTTAAAATTA TGTTTTAAAA |
| | TGGACTATCA |
| 201 | TATGCTTACC GTAACTTGAA AGTATTTCGA TTTCTTGGCT |
| | TTATATATCT |
| | sense |
| 251 | TGTGGAAAGG ACGAAACACC GTCGTACCGT ACGTAAGACAC |
| | loop |
| | TTTGTGTA |
| | [symbol] |
| 300 | [symbol] TTTTTT |

Site # 6 (812-832 bp) shNOX1 Construct Sequence: U6+1 promoter plus #6 shRNA sequence:

(SEQ ID NO: 7)

| | |
|---|---|
| | U6 promoter |
| 1 | GGATCCAAGG TCGGGCAGGA AGAGGGCCTA TTTCCCATGA |
| | TTCCTTCATA |
| 51 | TTTGCATATA CGATACAAGG CTGTTAGAGA GATAATTAGA |
| | ATTAATTTGA |
| 101 | CTGTAAACAC AAAGATATTA GTACAAAATA CGTGACGTAG |
| | AAAGTAATAA |
| 151 | TTTCTTGGGT AGTTTGCAGT TTTAAAATTA TGTTTTAAAA |
| | TGGACTATCA |
| 201 | TATGCTTACC GTAACTTGAA AGTATTTCGA TTTCTTGGCT |
| | TTATATATCT |
| | sense |
| 251 | TGTGGAAAGG ACGAAACACC GTCACGATCA TCCCACATCTC |
| | loop |
| | TTTGTGTA |
| | [symbol] |
| 300 | [symbol] TTTTTT |

Site # 8 (1171-1191 bp) shNOX1 Construct Sequence: U6+1 promoter plus #8 shRNA sequence:

(SEQ ID NO: 8)

| | |
|---|---|
| | U6 promoter |
| 1 | GGATCCAAGG TCGGGCAGGA AGAGGGCCTA TTTCCCATGA |
| | TTCCTTCATA |
| 51 | TTTGCATATA CGATACAAGG CTGTTAGAGA GATAATTAGA |
| | ATTAATTTGA |
| 101 | CTGTAAACAC AAAGATATTA GTACAAAATA CGTGACGTAG |
| | AAAGTAATAA |
| 151 | TTTCTTGGGT AGTTTGCAGT TTTAAAATTA TGTTTTAAAA |
| | TGGACTATCA |
| 201 | TATGCTTACC GTAACTTGAA AGTATTTCGA TTTCTTGGCT |
| | TTATATATCT |
| | sense |
| 251 | TGTGGAAAGG ACGAAACACC GAATATTGTT GTTCGAAAGCC |
| | loop |
| | TTTGTGTA |
| | [symbol] |
| 300 | [symbol] TTTTTT |

Cell Culture,transfection and stable clone selection: HT-29 colon cancer cell line was purchased from American Type Culture Collection (Manassas, Va.). HT-29 cells were maintained in McCoy's 5A (Irvine Scientific, Santa Ana, Calif.) with 2 mM L-glutamine and 10% fetal bovine serum. Cells were cultured in a 37° C. incubator in an atmosphere of 5% CO₂. Colon cancer cell line HT-29 was transfected with our shRNA expression vectors using Lipofectamine 2000 (Invitrogen, Carlsbad, Calif.) in a 1 ug DNA to 1.5 uL Lipofectamine 2000 ratio following the manufacturer's protocol. Transfection efficiency was 20-30%. Transiently transfected cells were sorted for GFP fluorescence on a MoFlo MLS (Dako-Cytomation, Fort Collins, Colo.) at the City of Hope flow cytometry core facility. Stable clones of HT-29 cells transfected with vectors containing #6 and scrambled targets were selected in media containing 700 ug/ml G418.

Doubling Time: Cell lines were harvested, counted and 5×10⁵ cells plated in T25 flasks in duplicate. After 72 hours cells were harvested and counted. Using the formula for exponential cell growth Nₜ=N₀2tf where t=72 hours, Nₜ=number of cells at 72 hours, N₀=initial number of cells and f=cell cycles per unit time; doubling time was calculated as t/3.3219(Log Nₜ-Log N₀). Average doubling time was calculated for the two replicates of each cell line. The experiment was repeated 3 times with similar results.

ROS detection with Flow Cytometry: Cells were harvested, washed with PBS and counted. One million cells were mixed with 1 mM DCF-DA (Molecular Probes, Eugene, Oreg., USA) in 10 μM final concentration. The samples were run, analyzed on MoFlo MLS (Dako-Cytomation, Fort Collins, Colo.) flow cytometer after 30 minutes incubation. Data was acquired using 488 nm laser excitation and 515 nm emission.

TUNEL Assay and Cell Cycle Analysis: Synchronized cells were harvested, fixed and permeabilized at 0, 24, 48 and 72 hours. Biotin-dUTP was incorporated into the 3′-strand breaks of apoptotic cells in a TdT-mediated dUTP nick end labeling (TUNEL) reaction. Cells were labeled with avidin-FITC for visualization by flow cytometry. Cells were labeled with propidium iodide for concomitant cell cycle analysis.

Flow cytometry was performed at the City of Hope flow cytometry core facility. The samples were acquired and analyzed on a MoFlo MLS flow cytometer (DAKO, Fort Collins, Colo.). Data was acquired using dual laser excitation. Scatter signals were acquired with a HeNe laser (Melles Groit, Carlsbad, Calif.). All fluorescence excitation was performed with 488 nm from an Innova-90 Argon laser (Coherent, Santa Clara, Calif.) at 500 mW. FITC emission was measured through a 530DF30 filter. Propidium iodide was measured through a 640EFLP filter. The two fluorescent signals were separated with a 580DRLP and a 630DRLP dichroic filter. All filters were purchased from Omega Optical (Brattleboro, Vt.). Data was acquired and analyzed with Summit software (DAKO).

Xenografts: Tumors were established by subcutaneous injection of 0.5×10⁶ cells bilaterally in the flanks of 6- to 8-week old male athymic mice (Charles River Laboratories, Wilmington, Mass.). Once tumors were palpable (10 days), bi-dimensional measurements were recorded for 17 days. Tumor volumes were calculated using the formula 0.5×L×W². Tumors were harvested and a portion of each tumor was segregated for fixation and embedding in paraffin. The remainder was preserved in RNAlater RNA stabilization reagent (QIAGEN, Valencia, Calif.) or frozen for subsequent extraction of RNA or protein for gene expression analysis.

CD31 Immunohistochemistry and blood vessel density analysis: Tumors were fixed in IHC zinc fixative (BD Pharmigen, San Diego, Calif.) and samples were stained with CD31 antibody (BD Pharmigen, San Diego, Calif.) at the City of Hope Anatomic Pathology Core Facility. The slides were analyzed with a Leica DM IRB microscope at 20× magnification. The number of blood vessels per field was quantified at five fields per section for eight tumors per condition by three investigators blinded to the samples identity.

Microarray analysis: The total RNA samples from the parental and stable transfected clone SA, 6A and the tumor samples from the xenograft experiment were analyzed on Affymetrix HG:U133A 2.0 arrays at UCI DNA & Protein MicroArray Facility, University of California, Irvine. All starting total RNA samples were quality assessed onto a RNA Lab-On-A-Chip (Caliper Technologies Corp., Mountain View, Calif.) that was evaluated on an Agilent Bioanalyzer 2100 (Agilent Technologies, Palo Alto, Calif.). Single-stranded, then double-stranded cDNA was synthesized 10 ug total RNA using the SuperScript Double-Stranded cDNA Synthesis Kit (Invitrogen Corp., Carlsbad, Calif.) and poly (T)-nucleotide primers. A portion of the resulting ds cDNA was used as a template to generate biotin-tagged cRNA from an in vitro transcription reaction (IVT), using the Affymetrix GeneChipe IVT Labeling Kit. The resulting biotin-tagged cRNA was fragmented (range 35-200 bases) following prescribed protocols (Affymetrix GeneChip® Expression Analysis Technical Manual). Subsequently, 10 ug of this fragmented target cRNA was hybridized at 45° C. with rotation for 16 hours (Affymetrix GeneChip® Hybridization Oven 640) to probe sets present on an Affymetrix HG:U133A 2.0 array. The GeneChip® arrays were washed and then stained (SAPE, streptavidin-phycoerythrin) on an Affymetrix Fluidics Station 450, followed by scanning on a GeneChip® Scanner 3000 The results were quantified and analyzed using GCOS 1.2 software (Affymetrix, Inc.) using default values (Scaling, Target Signal Intensity=500; Normalization, All Probe Sets; Parameters, all set at default values).

Quality assessment, preprocessing and statistical analysis of gene expression data were performed using the R statistical language and environment (http://www dot r-project dot org) and Bioconductor packages (http://www dot bioconductor dot org). The data analysis was improved using statistical models such as the Robust Multi-Chip Average (RMA) method. Genes with expression value less than 80 were excluded from further analysis. The Bioconductor package “limma” was used to identify the genes differentially expressed between 6A and SA samples. The R-contributed libraries “limma” (http://bioinf dot wehi dot edu dot au/limma/) provided the necessary routines for fitting linear models to microarray data that incorporate appropriate empirical Bayes smoothing of variances and for making multiple testing adjustments using the false discovery rate (FDR). We used FDR<0.05 and fold change>1.5 to identify differential expression. Genes passing above criteria between SA vs HT-29 samples were excluded because these gene expression changes might be due to transfection effects.

Hierarchical clustering using average linkage with the Pearson correlation was applied to differentially expressed gene expression data using the clustering features in GeneSpring v7.2 (Agilent, Palo Alto, Calif.). The genes showing altered expression were then categorized on the basis of their cellular components, biological processes, molecular functions and signaling pathways using the Database for Annotation, Visualization and Integrated Discovery 2.0 (DAVID 2.0, 17), GeneSpring, and the Ingenuity Pathways Analysis (Ingenuity, Mountain View, Calif.).

Western Blot Analysis: The cells were collected and washed once with phosphate buffer saline (PBS). Lysates were prepared by homogenizing cells in lysis buffer containing 10 mM Tris (pH8.0), 140 mM NaCl, 0.025% NaN₃, 1 mM EDTA, 1 mM phenylmethylsufonylfluoride (PMSF), 1% Triton X-100 and 50U/ml protease inhibitor cocktail. The lysates were left on ice before the centrifugation at 10,000×g for 30 min. Protein was measured using BioRad DC Protein Assay Kit (BioRad, Hercules, Ca). Whole cell lysates (20 μg protein per lane) were denatured by boiling in 2× Laemmli sample buffer and resolved by SDS-PAGE. The proteins were transferred to PVDF membrane (Amersham, Arlington Heights, Ill.) by electroblotting. The proteins were probed with antibodies from SantaCruz Biotechnologies Inc. (SantaCruz, Calif.). Blots were than incubated with HRP-linked secondary antibody followed by ECL⁺ dectection. Actin or tubulin was measured as the protein for loading control. The blots were quantified using ImageQuant software (Molecular Dynamics Inc., Sunnyvale, Calif.).

Statistical analysis: Values are presented as means±SEM. Student t-test was performed and values of p<0.05 were considered significant. Xenograft data was analyzed using 2-way ANOVA.

### Example 2

### NOX1 Subunit Expression in Colon Tumor and Adjacent Normal Tissue

NOX1 enzyme is highly specific for colon epithelial cells, generating superoxide in response to growth factors, cytokines, and other stimulants (Lambeth, 2002; Suh et al., 1999; Fukuyama et al., 2005). NOX1 might play a role in inflammatory response, be a part of host defense, stimulate cell proliferation and possibly be linked to tumorgenesis (Lambeth et al., 2000; Suh et al., 1999; Kikuchi et al., 2000).

We measured mRNA expression in 12 colon tumors and corresponding normal tissues. Expression of the NOX1 membrane bound catalytic core was significantly higher in cancerous tissue with an absolute mean value of 1714±446 (Mean±SEM) compared to 738±232 in adjacent normal tissue (p<0.05) (FIG. 1A). NOXO1 showed a highly significant (p<0.001) over expression in cancer tissues with absolute values 99±18 vs 22±5 in normal (FIG. 1B). NOXA1 expression was not significantly different in tumor and adjacent normal tissues (data not shown). p₂₂^{phox} expression was significantly higher in colon tumors (p<0.002) (data not shown). As a universal membrane bound subunit for members of the NOX gene family, p22^{phox} is an adapter protein that provides a binding site for the cytosolic regulatory proteins. Its significantly high level expression in tumor tissues is an indication of the increased enzyme activity. However, inhibition of this subunit would effect more then one gene, and cannot be used to study NOX1 gene regulation with siRNA in colon cancer. We concluded that inhibition of NOX1 or NOXO1 subunits would lead to a decrease in superoxide production, which might slow down cell proliferation.

### Example 3

### Down Regulation of NOX1 Gene Expression and Growth Rate of the Stable Clones

In our laboratory we used the small hairpin interfering RNA (shRNA) method of gene silencing to reduce Nox1 expression in colon cancer (Elbashir et al., 2001a. 2001b). Colon cancer cells (HT-29) were transiently transfected with the shRNA constructs, and stable clones were selected as described in the Methods.

NOX1 expression in the stable clones was measured through 20 passages, and was found to be significantly lower in stable clones 6A and 6C as compared to parental HT-29 cells or control clone SA (FIG. 2A). The mean value of NOX1 expression decreased from 158±25 in HT-29 and 126±16 in clone SA to 13±3 and 43±12 in clone 6A and 6C respectivel

A comparison of the growth rate of the clones as measured by doubling time assay showed a more than two fold increase in the doubling time of clone 6A and 6C, compared to the parental HT-29 cells and clone SA (FIG. 2B). Based on these results clone 6A was chosen for further analysis.

### Example 4

### ROS Production in Stable Clones

Previous studies have shown that colon cancer cells produce a significant amount of ROS (Droge, 2002; Szatrowski et al., 1991; Pemer et al., 2003). By silencing NOX1 gene expression we can saw a reduction in the amount of reactive oxygen species in HT-29 cells (FIG. 3.). The mean value of the fluorescence as measured by flow cytometry, shifted to the left from 3651.8 in parental cells to 2803.9 units in clone 6A. Clone SA seems to produce more ROS than parental cells, which might be explained as a host defense reaction against the expression vector.

### Example 5

### Cell Cycle Regulation in Stable Clones

Cell cycle regulation was studied in the stable clones by flow cytometry. Cells were synchronized and cell cycle progression was followed for 72 hrs. The HT-29 parental cells and clone SA underwent a regular cell cycle, while clone 6A showed slow cycle progression to the G2/M phase. At 24 hrs, only 5 % of the clone 6A cells progressed into the G2/M phase compared with 16-18% of the control cells. The parental and clone SA cells completed a cycle in approximately every 24-30 hrs, while clone 6A had a transient G1/S block requiring 72 hours to complete a cycle (FIG. 4.). This is in agreement with the doubling time results. The TUNEL assay did not show a significant difference between parental, clone SA and clone 6A cells for 72 hours, suggesting that apoptosis is not a major factor in the cell cycle arrest (data is not shown).

### Example 6

### HT-29 Xenograft in Athymic Mice

To study the in vivo effect of NOX1 silencing on tumor growth, we injected our clones in the flanks of athymic mice. Tumors established with clone 6A grew significantly slower than tumors established with clone SA or HT-29. On day 27, the average volume of 16 tumors in each group was 34±4 mm³ for clone 6A, 305±44 mm³ for HT-29 and 298±35 mm³ for clone SA (p<0.01, FIG. 5). The experiment was repeated twice with similar results. When clone 6A tumors were allowed to continue growing to day 41 the average volume was 158±17 mm³, similar to the volume of HT-29 tumors on day 20 (data not shown).

### Example 7

### Blood Vessel Density in the Tumors

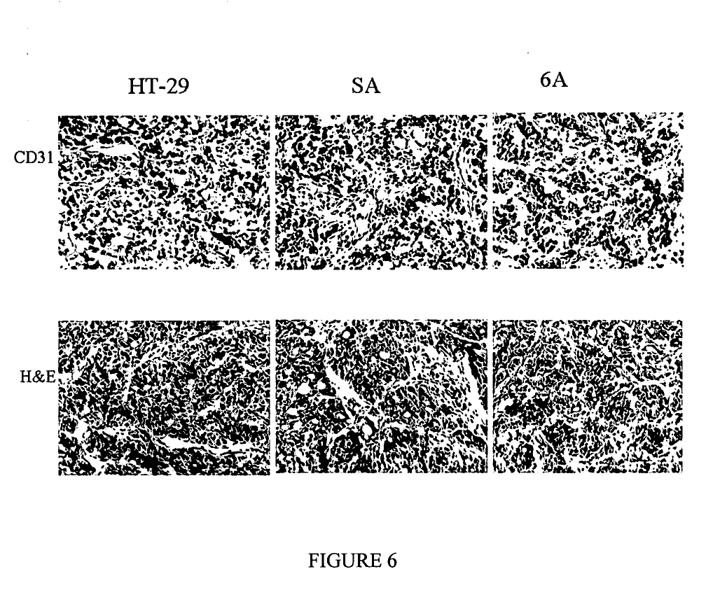

Harvested tumors were labeled with anti CD31 antibody to study the number of newly formed blood vessels. The number of vessels is decreased significantly from 81.9±1.4 (HT-29 parental) and 76.3±2.8 (SA) to 16.4±1.5 in clone 6A xenograft tumors. The diameter of the vessels was visibly smaller than in the controls (FIG. 6).

### Example 8

### Microarray Analysis of HT-29 Clones and Xenografts

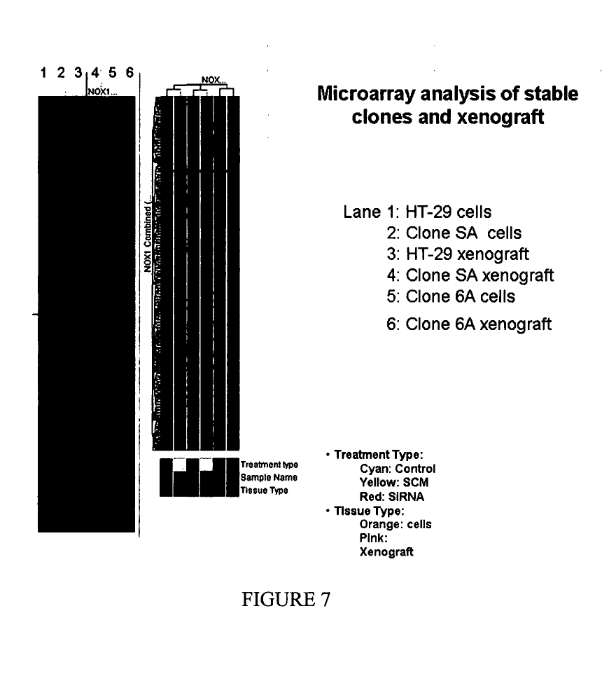

Total RNA was isolated from clone 6A, clone SA and parental HT-29 cells as well as from corresponding xenografts from triplicate experiments. Data was obtained for mean expression value, p value and fold change differences between parental cells and clones SA vs clone 6A and corresponding xenograft samples as described in the Methods. Results are shown in FIG. 7.

The complete list of genes from the microarray analysis was deposited in GEO database with accession number GSE4561, incorporated herein by reference.

Selected genes for which expression changed 2-fold or more in cells or in xenografts, and have known effects on cell cycle regulation, angiogenesis or are regulated by ROS were clustered according their molecular functions using GeneSpring (SilicoGenetics/Agilent, Palo Alto, Calif.) software (Tables 1 and 2). In these tables we have shown that the inhibition of Nox1 and subsequent reduction of ROS altered the expression of many genes suggesting a widespread effect on many biological functions, including repressed cell proliferation and angiogenesis.

TABLE 1
Down regulated genes in cells and xenograft sorted by funtion (C, SA vs si6A)

| | | Fold | Fold | |
|---|---|---|---|---|
| | | change in | change in | |
| Gene Symbol | Accession No | cells | xenograft | Gene description |
| FOS | BC004490 | NS | 2.5 | v-fos FBJ murine osteosarcoma viral oncogene homolog |

Cancer

| | | | | |
|---|---|---|---|---|
| FOS | BC004490 | NS | 2.5 | v-fos FBJ murine osteosarcoma viral oncogene homolog |
| MYB | NM_005375 | 4.6 | 4.3 | v-myb myeloblastosis viral oncogene homolog (avian) |
| MYC | NM_002467 | 2.0 | 2.1 | v-myc myelocytomatosis viral oncogene homolog (avian) |
| RAB26 | NM_014353 | NS | 3.3 | RAB26, member RAS oncogene family |
| TNS | NM_018274 | 6.0 | 3.8 | tensin /// tensin |
| VEGF | AF022375 | 1.7 | 3.6 | vascular endothelial growth factor |

Cell cycle

| | | | | |
|---|---|---|---|---|
| CDKN1A | NM_000389 | NS | 2.3 | cyclin-dependent kinase inhibitor 1A (p21, Cip1) |
| CDKN1C | N33167 | NS | 3.9 | cyclin-dependent kinase inhibitor 1C (p57, Kip2) |

Immunity

| | | | | |
|---|---|---|---|---|
| AZGP1 | D90427 | 12.8 | 13.9 | alpha-2-glycoprotein 1, zinc |
| CCL14///15 | AF031587 | 30.1 | 6.5 | chemokine (C-C motif) ligand 14 /// 15 |
| CCL20 | NM_004591 | NS | 4.7 | chemokine (C-C motif) ligand 20 |
| CEACAM5 | NM_004363 | NS | 7.7 | carcinoembryonic antigen-related cell adhesion molecule 5 |
| CEACAM7 | NM_006890 | NS | 6.6 | carcinoembryonic antigen-related cell adhesion molecule 7 |
| F5 | NM_000130 | 8.7 | 1.6 | coagulation factor V (proaccelerin, labile factor) |
| GPA33 | NM_005814 | NS | 6.4 | glycoprotein A33 (transmembrane) |
| TBXAS1 | NM_030984 | 4.1 | 2.9 | thromboxane A synthase 1 |

Enzymes

| | | | | |
|---|---|---|---|---|
| AKR1B10 | NM_020299 | 28.4 | 16.1 | aldo-keto reductase family 1, member B10 |
| AKR1C1 | NM_001353 | 5.1 | 2.3 | aldo-keto reductase family 1, member C1 |
| ANPEP | NM_001150 | 2.1 | 23.1 | alanyl (membrane) aminopeptidase, CD13, p150) |
| ASNS | NM_001673 | 2.5 | 4.1 | asparagine synthetase |
| CA12 | NM_001218 | 11.9 | 11.5 | carbonic anhydrase XII |
| CA9 | NM_001216 | NS | 3.8 | carbonic anhydrase IX |
| DDC | NM_000790 | 1.6 | 14.0 | dopa decarboxylase (aromatic L-amino acid decarboxylase) |
| DPP4 | NM_001935 | 2.8 | 8.6 | dipeptidylpeptidase 4 (CD26) |
| DUSP5 | U16996 | NS | 3.4 | dual specificity phosphatase 5 |
| GPD1 | NM_005276 | NS | 3.0 | glycerol-3-phosphate dehydrogenase 1 (soluble) |
| GPX3 | AW149846 | NS | 3.5 | glutathione peroxidase 3 (plasma) |
| GSTA4 | NM_001512 | 4.4 | 3.3 | glutathione S-transferase A4 |
| HK2 | AI761561 | NS | 4.1 | hexokinase 2 |
| HMOX1 | NM_002133 | 1.9 | 1.7 | heme oxygenase (decycling) 1 |
| HSD17B2 | NM_002153 | NS | 12.7 | hydroxysteroid (17-beta) dehydrogenase 2 |
| IDH2 | AU151428 | 2.5 | 3.6 | isocitrate dehydrogenase 2 (NADP+), mitochondrial |
| NOX1 | NM_007052 | 6.2 | 19.2 | NADPH oxidase 1 |
| PAPSS2 | NM_004670 | 8.3 | 3.2 | 3′-phosphoadenosine 5′-phosphosulfate synthase 2 |
| SERPINE2 | AL541302 | NS | 20.9 | serine (or cysteine) proteinase inhibitor, clade E2 |
| SHMT2 | AW190316 | 2.4 | 3.1 | serine hydroxymethyltransferase 2 (mitochondrial) |
| SI | NM_001041 | NS | 15.7 | sucrase-isomaltase (alpha-glucosidase) |
| SMP3 | NM_025163 | 3.2 | 12.7 | SMP3 mannosyltransferase |
| ST6GAL1 | AI743792 | 3.9 | 3.9 | ST6 beta-galactosamide alpha-2,6-sialyltranferase 1 |

Nucleic acid binding proteins

| | | | | |
|---|---|---|---|---|
| ADM | NM_001124 | NS | 7.0 | adrenomedullin |
| BGN | AA845258 | 5.9 | 2.8 | biglycan///serologically defined colon cancer antigen 33 |
| BHLHB2 | NM_003670 | NS | 3.0 | basic helix-loop-helix domain containing, class B, 2 |
| CREB3L2 | BE675139 | 3.2 | 1.8 | cAMP responsive element binding protein 3-like 2 |
| FOXA2 | AB028021 | NS | 5.3 | forkhead box A2 |
| GNAI1 | AL049933 | 4.8 | 2.3 | guanine nucleotide binding protein (G protein), |
| KCNH2 | AB044806 | 1.9 | 3.2 | potassium voltage-gated channel, subfamily H member 2 |
| LGALS4 | NM_006149 | 10.7 | 2.1 | lectin, galactoside-binding, soluble, 4 (galectin 4) |
| MGA | BE502432 | 4.8 | 3.9 | MAX gene associated |
| NR1I2 | NM_003889 | NS | 3.7 | nuclear receptor subfamily 1, group I, member 2 |
| TNRC9 | AK025084 | 48.1 | 3.2 | trinucleotide repeat containing 9 |

Signal transduction

| | | | | |
|---|---|---|---|---|
| ANXA10 | AF196478 | 36.4 | NS | annexin A10 |
| ANXA13 | NM_004306 | NS | 29.6 | annexin A13 |
| CTGF | M92934 | 3.1 | 4.2 | connective tissue growth factor |
| CXCR4 | L01639 | 12.0 | 45.1 | chemokine (C-X-C motif) receptor 4 |
| EFNA1 | NM_004428 | NS | 2.8 | ephrin-A1 |
| FGFR3 | NM_000142 | 12.4 | 1.9 | fibroblast growth factor receptor 3 (thanatophoric dwarfism) |
| FGFR4 | AF202063 | 2.8 | 1.7 | fibroblast growth factor receptor 4 |
| IGFBP2 | NM_000597 | NS | 17.4 | insulin-like growth factor binding protein 2, 36 kDa |
| INSR | AA485908 | NS | 3.2 | Insulin receptor |
| JAG1 | U77914 | 2.1 | 2.1 | jagged 1 (Alagille syndrome) |
| LGALS2 | NM_006498 | NS | 8.5 | lectin, galactoside-binding, soluble, 2 (galectin 2) |
| NDRG1 | NM_006096 | NS | 17.3 | N-myc downstream regulated gene 1 |

Others

| | | | | |
|---|---|---|---|---|
| TFPI | AF021834 | 4.7 | 2.7 | tissue factor pathway inhibitor (coagulation inhibitor) |
| TPM1 | M19267 | 2.5 | 2.8 | Tropomyosin 1 (alpha) |

Structural

| | | | | |
|---|---|---|---|---|
| AKAP7 | AL137063 | 13.5 | 67.3 | A kinase (PRKA) anchor protein 7 |
| CFTR | NM_000492 | NS | 3.2 | cystic fibrosis transmembrane conductance regulator |
| ECM1 | U65932 | NS | 3.3 | extracellular matrix protein 1 |
| ITPR2 | NM_002223 | 2.9 | 5.6 | inositol 1,4,5-triphosphate receptor, type 2 |
| SEPP1 | NM_005410 | NS | 14.1 | selenoprotein P, plasma, 1 |

Transport proteins

| | | | | |
|---|---|---|---|---|
| ATP2A3 | AA877910 | 2.2 | 3.5 | ATPase, Ca++ transporting, ubiquitous |
| ATP7B | NM_000053 | 2.3 | 6.0 | ATPase, Cu++ transporting, beta polypeptide |
| FABP1 | NM_001443 | NS | 15.6 | fatty acid binding protein 1, liver |
| HEPH | NM_014799 | NS | 85.8 | hephaestin, ferroxidase |
| KCTD12 | AI718937 | NS | 17.3 | potassium channel tetramerisation domain containing 12 |
| SLC11A2 | AF046997 | NS | 3.1 | solute carrier family 11 (H⁺-coupled metal ion transporters)2 |
| SLC26A2 | AI025519 | NS | 3.7 | solute carrier family 26 (sulfate transporter), member 2 |
| SLC27A2 | NM_003645 | 1.7 | 6.9 | solute carrier family 27 (fatty acid transporter), member 2 |
| SLC3A1 | NM_000341 | NS | 44.5 | solute carrier family 3 (amino acid transporters) 1 |
| SLC43A1 | NM_003627 | 5.5 | 4.5 | solute carrier family 43, member 1 (neutral amino acid) |
| SLC5A1 | NM_000343 | NS | 8.8 | solute carrier family 5 (sodium/glucose cotransporter) 1 |
| TFRC | NM_003234 | 2.3 | 3.2 | transferrin receptor (p90, CD71) |

Unclassified

| | | | | |
|---|---|---|---|---|
| ABP1 | NM_001091 | NS | 9.4 | amiloride binding protein 1 (amine oxidase (Cu-containing)) |
| AGT | NM_000029 | 2.8 | 2.4 | angiotensinogen (serine (or cysteine) proteinase inhibitor) |
| DDIT4 | NM_019058 | NS | 8.4 | DNA-damage-inducible transcript 4 |
| ENG | NM_000118 | 2.8 | 1.8 | endoglin (Osler-Rendu-Weber syndrome 1) |
| MPRG | NM_017705 | 2.4 | 3.9 | membrane progestin receptor gamma |
| SPRY2 | NM_005842 | 2.7 | 3.8 | sprouty homolog 2 (Drosophila) |

TABLE 2
Up regulated genes in cells and xenograft sorted by function (C, SA vs si6A)

| | | Fold | Fold | |
|---|---|---|---|---|
| | | change | change in | |
| Gene Symbol | Accession No | in cells | xenograft | Gene description |
| — | AW474434 | 8.4 | NS | — |

Apoptosis

| | | | | |
|---|---|---|---|---|
| — | AW474434 | 8.4 | NS | — |
| CASP1 | U13700 | NS | 2.4 | caspase 1, apoptosis-related cysteine protease |
| CD14 | NM_000591 | 7.3 | 4.0 | CD14 antigen |
| TNFSF10 | NM_003810 | 8.7 | NS | tumor necrosis factor (ligand) superfamily, member 10 |

Cancer

| | | | | |
|---|---|---|---|---|
| MYBL1 | AW592266 | NS | 6.5 | v-myb myeloblastosis viral oncogene homolog like 1 |
| PIAS3 | NM_006099 | 1.6 | 3.1 | protein inhibitor of activated STAT, 3 |
| RARRES1 | NM_002888 | 7.0 | NS | retinoic acid receptor responder (tazarotene induced) 1 |
| RARRES3 | NM_004585 | NS | 4.0 | retinoic acid receptor responder (tazarotene induced) 3 |
| RUNX1 | BF432501 | 2.4 | 2.3 | runt-related transcription factor 1 (aml1 oncogene) |

Cell cycle

| | | | | |
|---|---|---|---|---|
| CDKN2C | NM_001262 | 2.2 | 3.5 | cyclin-dependent kinase inhibitor 2C (p18, inhibits CDK4) |
| CDKN2D | U20498 | 1.6 | 1.7 | cyclin-dependent kinase inhibitor 2D (p19, inhibits CDK4) |
| TGFB1 | BC001830 | 4.2 | 2.6 | transforming growth factor beta 1 induced transcript 1 |

Chaperone

| | | | | |
|---|---|---|---|---|
| ARHGDIB | NM_001175 | 2.4 | 2.4 | Rho GDP dissociation inhibitor (GDI) beta |
| CD74 | K01144 | 3.3 | NS | CD74 antigen (class II antigen-associated) |
| EPB41L1 | AL121895 | 2.6 | 3.7 | erythrocyte membrane protein band 4.1-like 1 |
| NEDD9 | AL136139 | 4.5 | 1.7 | neural precursor cell expressed, down-regulated 9 |
| SVIL | NM_003174 | 3.6 | 1.7 | supervillin |

Enzymes

| | | | | |
|---|---|---|---|---|
| ALDH1A3 | AF198444 | 3.6 | NS | Aldehyde dehydrogenase 1 family, member A3 |
| ALOX5 | NM_000698 | 4.5 | 2.1 | arachidonate 5-lipoxygenase |
| DUSP10 | N36770 | 3.3 | 2.2 | dual specificity phosphatase 10 |
| HPGD | NM_000860 | 4.7 | 2.0 | hydroxyprostaglandin dehydrogenase 15-(NAD) |
| INPP4B | NM_003866 | 5.1 | NS | inositol polyphosphate-4-phosphatase, type II, 105 kDa |
| KYNU | D55639 | 3.1 | 6.6 | kynureninase (L-kynurenine hydrolase) |
| PLAU | NM_002658 | 2.4 | 2.6 | plasminogen activator, urokinase |
| PPP2R3A | AL389975 | 3.4 | 1.7 | protein phosphatase 2, regulatory subunit B″, alpha |
| PTGS2 | NM_000963 | 7.0 | NS | prostaglandin-endoperoxide synthase 2 (cox-2) |
| TGM2 | AL031651 | NS | 7.2 | transglutaminase 2 (C polypeptide) |

Immunity proteins

| | | | | |
|---|---|---|---|---|
| ALCAM | BF242905 | 2.4 | 7.9 | Activated leukocyte cell adhesion molecule |
| COCH | AA669336 | 3.5 | 2.4 | coagulation factor C homolog, cochlin |
| IFI44L | NM_006820 | NS | 3.8 | interferon-induced protein 44-like |
| IFI16 | AF208043 | NS | 5.2 | interferon, gamma-inducible protein 16 |
| KIAA0992 | NM_016081 | 2.5 | 4.5 | palladin |

Nucleic acid binding proteins

| | | | | |
|---|---|---|---|---|
| ARL7 | BC001051 | 2.4 | 4.6 | ADP-ribosylation factor-like 7 |
| ELK3 | AW575374 | 1.5 | 2.5 | ELK3, ETS-domain protein (SRF accessory protein 2) |
| FOXC1 | AU145890 | 3.3 | 2.4 | Forkhead box C1 |
| TRIM29 | NM_012101 | 3.1 | 1.5 | tripartite motif-containing 29 |
| VGLL1 | NM_016267 | 3.9 | 1.7 | vestigial like 1 (Drosophila) |
| ZNF211 | NM_006385 | 3.1 | 2.1 | zinc finger protein 211 |

Signal transduction

| | | | | |
|---|---|---|---|---|
| AXL | NM_021913 | 37.9 | 5.3 | AXL receptor tyrosine kinase |
| GAS6 | NM_000820 | 12.6 | 2.7 | growth arrest-specific 6 |
| GPR87 | NM_023915 | NS | 6.0 | G protein-coupled receptor 87 |
| IGFBP6 | NM_002178 | NS | 3.2 | insulin-like growth factor binding protein 6 |
| NMU | NM_006681 | 8.4 | 7.7 | neuromedin U |
| NRP1 | BE620457 | NS | 5.1 | neuropilin 1 |
| SOSTDC1 | AI927000 | 7.3 | NS | sclerostin domain containing 1 |
| TACSTD2 | J04152 | 7.5 | NS | tumor-associated calcium signal transducer 2 |
| WNT11 | NM_004626 | NS | 3.0 | wingless-type MMTV integration site family, 11 |

Others

| | | | | |
|---|---|---|---|---|
| CDH11 | D21254 | NS | 3.5 | cadherin 11, type 2, OB-cadherin (osteoblast) |
| CST1 | NM_001898 | 29.5 | NS | cystatin SN |
| CST4 | NM_001899 | 3.2 | NS | cystatin S |
| CST6 | NM_001323 | 8.5 | 6.4 | cystatin E/M |
| RGC32 | NM_014059 | 4.0 | NS | response gene to complement 32 |
| S100A2 | NM_005978 | 5.6 | NS | S100 calcium binding protein A2 |
| TPBG | NM_006670 | NS | 3.4 | trophoblast glycoprotein |

Structural proteins

| | | | | |
|---|---|---|---|---|
| CAV1 | NM_001753 | 2.9 | 2.3 | caveolin 1, caveolae protein, 22 kDa |
| MMP14 | X83535 | NS | 2.3 | matrix metalloproteinase 14 (membrane-inserted) |
| MUC16 | NM_024690 | NS | 7.9 | mucin 16 |
| ADAM28 | NM_021778 | 5.0 | 1.7 | a disintegrin and metalloproteinase domain 28 |
| KRT13 | NM_002274 | 11.2 | 12.3 | keratin 13 |

Transport proteins

| | | | | |
|---|---|---|---|---|
| AP1S2 | NM_003916 | 3.8 | 1.9 | adaptor-related protein complex 1, sigma 2 subunit |
| CLIC5 | AL049313 | 3.8 | 1.7 | Chloride intracellular channel 5 |
| SLC39A8 | AB040120 | NS | 3.5 | solute carrier family 39 (zinc transporter), member 8 |
| SLC6A14 | NM_007231 | NS | 12.0 | solute carrier family 6 (neurotransmitter transporter), 14 |
| SLC01B3 | NM_019844 | NS | 9.3 | solute carrier organic anion transporter family, 1B3 |
| TCN1 | NM_001062 | 12.1 | 3.8 | transcobalamin I (vitamin B12 binding protein) |
| TRPC1 | NM_003304 | NS | 3.7 | transient receptor potential cation channel, C, 1 |

Unclassified

| | | | | |
|---|---|---|---|---|
| C10orf116 | BC004471 | 4.1 | 2.8 | chromosome 10 open reading frame 116 |
| C10orf81 | NM_024889 | NS | 3.9 | chromosome 10 open reading frame 81 |
| C6orf15 | NM_014070 | 4.1 | NS | chromosome 6 open reading frame 15 |
| CNN3 | NM_001839 | 12.0 | 2.1 | calponin 3, acidic |
| DBP | U79283 | 5.9 | 1.5 | D site of albumin promoter (D-box) binding protein |
| DOC1 | NM_014890 | 4.2 | NS | downregulated in ovarian cancer 1 |
| DOCK9 | AL576253 | 3.3 | 3.8 | dedicator of cytokinesis 9 |
| EHD2 | AI417917 | 3.4 | 2.3 | EH-domain containing 2 |
| FLJ11259 | NM_018370 | 2.5 | 14.4 | hypothetical protein FLJ11259 |
| PER3 | NM_016831 | 3.4 | 2.7 | period homolog 3 (Drosophila) |
| RALGPS1 | NM_014636 | 3.0 | NS | Ral GEF with PH domain and SH3 binding motif 1 |
| SACS | AI932370 | NS | 4.1 | spastic ataxia of Charlevoix-Saguenay (sacsin) |
| SCEL | NM_003843 | NS | 5.7 | sciellin |
| SHANK2 | BF435773 | 4.8 | 2.1 | SH3 and multiple ankyrin repeat domains 2 |
| TMEPAI | NM_020182 | 4.9 | 2.6 | transmembrane, prostate androgen induced RNA |
| TRIM22 | AA083478 | NS | 5.2 | tripartite motif-containing 22 |
| TXNIP | NM_006472 | 3.4 | NS | thioredoxin interacting protein |
| UPK3B | NM_030570 | NS | 5.8 | uroplakin 3B |
| WFDC2 | NM_006103 | NS | 3.2 | WAP four-disulfide core domain 2 |
| PP1665 | AL041124 | 6.4 | NS | hypothetical protein PP1665 |

### Example 9

### Validation of the Microarray Data with Real-Time PCR and Western-Blot

We used real time PCR to validate the microarray data of selected genes that might be involved in carcinogenesis. Table 3A shows expression data for HT-29 parental, clone SA and 6A cells and Table 3B summarize the results obtained from xenograft samples in each tumor group as representative of 2-3 independent experiments. Expression of the target gene, NOXI was significantly (p<0.001) inhibited in clone 6A (13±2) as compared to HT-29 (158±20) or SA (140±12) cells and clone 6A (26±3) versus HT-29 (381±28) or SA (580±48) xenograft samples (Table 3).

TABLE 3
Gene expression in stable clones and xenograft by real-Time PCR

| | | | | |
|---|---|---|---|---|
| | | | | p value |
| | | | | (HT or SA |
| Gene | HT-29 | Scrambled A | Clone 6 A | vs si6A) |

A Cells
Down-regulated

| | | | | |
|---|---|---|---|---|
| CCL14/15 | 265 ± 79 | 148 ± 48 | 2 ± 1 | <0.05 |
| c-MYB | 108 ± 7 | 63 ± 10 | 13 ± 2 | <0.005 |
| c-MYC | 956 ± 225 | 758 ± 154 | 287 ± 75 | <0.05 |
| CXCR4 | 5115 ± 1170 | 2240 ± 87 | 7 ± 2 | <0.005 |
| FGFR3 | 348 ± 53 | 313 ± 27 | 15 ± 3 | <0.05 |
| VEGF-A | 402 ± 70 | 473 ± 79 | 252 ± 37 | <0.05 |
| HMOX1 | 73 ± 25 | 104 ± 47 | 30 ± 12 | <0.05 |
| NOX1 | 158 ± 20 | 140 ± 12 | 13 ± 2 | <0.001 |

Up-regulated

| | | | | |
|---|---|---|---|---|
| AXL | 1 ± 0.3 | 1 ± 0.1 | 89 ± 11 | <0.005 |
| CDKN2C | 3 ± 1 | 2 ± 0.3 | 6 ± 1 | <0.05 |
| CST1 | 10 ± 1 | 10 ± 5 | 603 ± 157 | <0.05 |
| GAS6 | 1 ± 0.3 | 1 ± 0.1 | 11 ± 8 | <0.05 |
| TGFB1 | 172 ± 24 | 205 ± 37 | 623 ± 88 | <0.005 |

No significant change

| | | | | |
|---|---|---|---|---|
| ADM | 54 ± 12 | 26 ± 4 | 56 ± 10 | N.S |
| CAT | 642 ± 31 | 288 ± 82 | 534 ± 16 | N.S. |
| c-FOS | 540 ± 103 | 190 ± 20 | 328 ± 82 | N.S |
| HIF1a | 109 ± 25 | 70 ± 14 | 119 ± 34 | N.S. |

B Xenograft
Down-regulated

| | | | | |
|---|---|---|---|---|
| ADM | 151 ± 15 | 67 ± 9 | 19 ± 3 | <0.005 |
| CCL14/15 | 431 ± 38 | 424 ± 48 | 36 ± 13 | <0.005 |
| c-MYB | 47 ± 7 | 45 ± 13 | 6 ± 3 | <0.05 |
| c-MYC | 563 ± 48 | 704 ± 115 | 246 ± 36 | <0.05 |
| c-FOS | 122 ± 25 | 103 ± 34 | 47 ± 18 | <0.05 |
| CXCR4 | 40 ± 5 | 36 ± 5 | 1 ± 0.2 | <0.005 |
| HMOX1 | 43 ± 3 | 38 ± 4 | 3 ± 1 | <0.005 |
| NOX1 | 381 ± 28 | 580 ± 48 | 26 ± 3 | <0.005 |
| VEGF-A | 796 ± 98 | 751 ± 62 | 247 ± 35 | <0.005 |

Up-regulated

| | | | | |
|---|---|---|---|---|
| AXL | 11 ± 1 | 4 ± 1 | 40 ± 6 | <0.005 |
| CDKN2C | 2 ± 0.2 | 2 ± 0.3 | 6 ± 2 | <0.05 |
| CST1 | 5 ± 1 | 2 ± 0.3 | 11 ± 3 | <0.05 |
| GAS6 | 3 ± 1 | 3 ± 0.2 | 18 ± 3 | <0.005 |
| TGFB1 | 303 ± 31 | 522 ± 26 | 2124 ± 281 | <0.005 |

No significant change

| | | | | |
|---|---|---|---|---|
| CAT | 836 ± 126 | 494 ± 135 | 518 ± 78 | N.S. |
| FGFR3 | 441 ± 37 | 552 ± 61 | 331 ± 74 | N.S |
| HIF1a | 43 ± 7 | 24 ± 5 | 16 ± 6 | N.S |

Values (Mean ± SEM) represent the ratio of target gene expression to β-Actin expression ×10⁻⁴. The data represents 3 independent experiments (N = 3-15).

c-MYC, c-FOS and c-MYB, genes known to play a role in cell cycle regulation and proliferation, showed significant ( p<0.005) down regulation in clone 6A and 6A xenograft. Expression of TGFβ1 showed significant (p<0.005) up regulation in clone 6A cells and tissue samples (Table 3 A-B). TGFβ1 regulates the expression of two groups of cyclin-dependent kinase inhibitors, INK4 and Cip/Kip. CDKN2C (p18) a member of the INK4 gene family and an inhibitor of CDK4 was up regulated both in clone 6A cells and xenograft (Table 3 A-B). Cell cycle proteins were studied in stable clones serum starved for 22 hrs to synchronize the cell cycle. Proteins regulating Gl/S transition were studied by western-blot analysis (FIG. 8).

As expected, CDK4 protein was down regulated in clone 6A cells up to 72 hrs due to over expression of CDK4 inhibitor CDKN2C. CDK2 and cyclin E proteins were lower in clone si6A at 0 hr than in parental and clone SA cells, but increased as the cell cycle progressed. The increase in cyclin D1 protein and decrease in p21protein in clone 6A in early stages of cell cycle indicate that cyclin D1 might override the inhibiting effect of p18.

Selected genes that play a key role in angiogenesis were validated with real-time PCR. VEGF-A expression decreased 2-fold in clone 6A (p<0.05). More significantly, expression of VEGF-A was decreased 3.5-fold in clone 6A xenograft indicating the important role of the tumor microenvironment in regulation of this gene (p<0.005). Recently, Iimuro et.al. showed a connection between VEGF induced angiogenesis and adenomedullin (ADM), a vasodilating peptide (Iimuro et al., 2004). They demonstrated enhanced capillary formation and increased blood flow in mice upon co-administration of VEGF and ADM. Our data was consistent with their findings; we observed 7-fold down-regulation of ADM in clone 6A xenograft (p<0.005, Table 3B).

Hemeoxygenase (HMOX1) gene, which is known to be regulated by ROS, showed a 2-fold inhibition in clone 6A compared to parental HT-29 or clone SA (p<0.05) and an eightfold reduction in clone 6A xenograft (p<0.005) by real-time PCR. However, the expression of HIFla, gene up-stream of HMOX1, was not significantly changed in a clone 6A cells or xenograft.

Chemokine receptor (CXCR4) and ligand (CCL14/15) play a very important role in oxygen-dependent regulation of angiogenesis (Schioppa et al., 2003; Hwang et al., 2004). CXCR4 was decreased in clone 6A cells 320-fold and 35-fold in clone 6A xenograft, respectively. CCL14/15 showed a 70-fold down-regulation in clone 6A and a 12-fold down-regulation in xenografts (Table 3A-B). Our data suggest that inhibition of the ROS production in clone 6A by shRNA silencing of NOX1 can attenuate angiogenesis.

AXL, a receptor tyrosine kinase, and its ligand GAS6 are highly over- expressed in clone si6A compared to HT-29 or clone SA (88 and 120-fold respectively) and moderately over-expressed in xenograft tissues (6-fold), p<0.005 (Table 3A-B). The role of these genes in the regulation of tumor growth and in the oxygen-dependent regulation of angiogenesis are not fully understood yet.

In addition of to validation by real-time RT-PCR protein levels of selected genes were measured by Western-blot analysis (FIG. 9).

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Ambasta, R. K. et al. (2004). Direct interaction of the novel Nox proteins with p22phox is required for the formation of a functionally active NADPH oxidase. J Biol Chem 279(44):45935-45941.

Arbiser, J. L. et al. (2002). Reactive oxygen generated by Nox1 triggers the angiogenic switch. Proc Natl Acad Sci USA 99(2):715-720.

Amarzguioui, M. et al. (2003). Tolerance for Mutation and Chemical Modifications in a siRNA. Nucleic Acids Research 31:589-595.

Arnold, R. S. et al. (2001). Hydrogen peroxide mediates the cell growth and transformation caused by the mitogenic oxidase Nox1. Proc Natl Acad Sci USA 98(10):5550-5555.

Babior, B. M. (1999). NADPH Oxidase: An Update. Blood 93(5):1464-1476.

Banfi, B. et al. (2000). A Mammalian H⁺ Channel Generated Through Alternative Splicing of the NADPH Oxidase Homolog NOH-1. Science 287:138-142.

Banfi, B. et al. (2001). A Ca2+-activated NADPH Oxidase in Testis, Spleen, and Lymph Nodes. J Biol Chem 276(40):37594-37601.

Banfi, B. et al. (2003). Two novel proteins activate superoxide generation by the NADPH oxidase NOX1. J Biol Chem 278(6):3510-3513.

Berberat, P. O. et al. (2005). Inhibition of Heme Oxygenase-1 Increases Responsiveness of Pancreatic Cancer Cells to Anticancer Treatment. Clin Can Res 11(10):3790-3798.

Bernstein, E. et al. (2001) Role for a bidentate ribonuclease in the initiation step of RNA interference. Nature 409:363-366.

Bernstein, E. et al. (2001). The rest is silence. Rna 7(11):1509-1521.

Bertrand, E. et al. (1997). The expression cassette determines the functional activity of ribozymes in mammalian cells by controlling their intracellular localization. RNA 3:75-88.

Brummelkamp, T. R. et al. (2002). A system for stable expression of short interfering RNAs in mammalian cells. Science 296(5567):550-553.

Castanotto, D. et al. (2002). Functional siRNA expression from transfected PCR products. RNA 8:1454-1460.

Cheng, G. and Lambeth, J. D. (2004). NOXO1, regulation of lipid binding, localization, and activation of Nox1 by the Phox homology (PX) domain. J Biol Chem 279(6):4737-4742.

Cheng, G. and Lambeth, J. D. (2005). Alternative mRNA splice forms of NOXO1: Differential tissue expression and regulation of Nox1 and Nox3. Gene 356:118-126.

Cheng, G. et al. (2001). Homologs of gp91phox: cloning and tissue expression of Nox3, Nox4, and Nox5. Gene 269(1-2): 131-140.

Chen, Y. et al. (2003). Down-Regulation of CXCR4 by inducible small interfering RNA inhibits breast cell invasion in Vitro. Cancer Research 63:4801-4804.

Droge, W. (2002). Free Radicals in the Physiological Control of Cell Function. Physiological Reviews 82:47-95.

Dupuy, C. et al. (1999). Purification of a Novel Flavoprotein Involved in the Thyroid NADPH Oxidase. J Biol Chem 274(52):37265-37269.

Eckstein, F. (2000). Phosphorothioate oligodeoxynucleotides: what is their origin and what is unique about them? Antisense Nucleic Acid Drug Dev 10:117-21.

Elbashir, S. M. et al. (2001b). RNA interference is mediated by 21- and 22-nucleotide RNAs. Genes & Dev 15:188-200.

Elbashir, S. M. et al. (2001a). Duplexes of 21-nucleotide RNAs mediate RNA interference in cultured mammalian cells. Nature 411:494-498.

Fiebeler, A. et al. (2004). Growth Arrest Specific protein 6/AXL Signaling in human Inflammatory Renal Diseases. Am J Kidney Diseases 43(2):286-295.

Fukuyama, M. et al. (2005). Overexpression of a novel superoxide-producing enzyme, NADPH oxidase 1, in adenoma and well differentiated adenocarcinoma of the human colon. Cancer Letters 221:97-104.

Gallicchio M et al. (2005). Inhibition of vascular endothelial growth factor receptor-2-mediated endothelial cell activation by Ax1 tyrosine kinase receptor. Blood 105(5):1970-1976.

Geiszt, M. et al. (2000). Identification of renox, an NAD(P)H oxidase in kidney. Proc Natl Acad Sci USA 97:8010-8014.

Good, P. D. et al. (1997). Expression of small, therapeutic RNAs in human cell nuclei. Gene Ther 4:45-54.

Goyal, P. et al. (2004). Upregulation of NAD(P)H oxidase 1 in hypoxia activates Hypoxia- inducible factor I via increase in reactive oxygen species. Free Radical Biology & Medicine 36(10):1279-1288.

Grunewald, M. et al. (2006). VEGF-Induced Adult Neovascularization: Recruitment, Retention, and Role of Accessory Cells. Cell 124:175-189.

Guleng B. et al. (2005). Blocade of the stromal cell-derived factor-1/CXCR4 axis attenuates in vivo tumor growth by inhibiting angiogenesis in a vascular endothelial growth factor-independent manner. Cancer Research 65(13):5864-5871.

Hamilton, A. et al. (2002). Two classes of short interfering RNA in RNA silencing. EMBO Journal 21:4671-4679.

Hammond, S. M., et al. (2001). Argonaute2, a link between genetic and biochemical analyses of RNAi. Science 293(5532):1146-1150.

Hanna, I. R. et al. (2004). Functional association of NOX1 with p22phox in vascular smooth muscle cells. Free Radical Biology & Medicine 37(10):1542-1549.

Harborth, J. et al. (2003). Sequence, chemical, and structural variation of small interfering RNAs and short hairpin RNAs and the effect on mammalian gene silencing. Antisense Nucleic Acid Drug Dev 13:83-105.

Heidel, J. D. et al. (2004). Lack of interferon response in animals to naked siRNAs. Nat Biotechnol 22:1579-1582.

Herdewijn, P. (2000). Heterocyclic modifications of oligonucleotides and antisense technology. Antisense Nucleic Acid Drug Dev 10:297-310.

Herman, J. G. et al. (1996). Methylation-Specific PCR: A Novel PCR Assay for Methylation Status of CpG Islands. Proc Natl Acad Sci USA 93:9821-9826.

Hodge, D. R. et al. (2005). The role of IL-6 and STAT3 in inflammation and cancer. Eur J Cancer 41:2502-2512.

Holen, T. et al. (2002). Positional effects of short interfering RNAs targeting the human coagulation trigger tissue factor. Nucleic Acids Res 30:1757-1766.

Holland S. J. et al. (2005). Multiple Roles for the Receptor Tyrosine Kinase Ax1 in Tumor Formation. Cancer Research 65(20):9294-9303.

Hornung, V. et al. (2005). Sequence-specific potent induction of IFN-alpha by short interfering RNA in plasmacytoid dendritic cells through TLR7. Nature Med 11:263-270.

Hu-Lieskovan, S. et al. (2005). Sequence-specific knockdown of EWS-FLII by targeted, non-viral delivery of siRNA inhibits tumor growth in a murine model of metastatic Ewing's sarcoma. Cancer Res 65(19):8984-92.

Hwang, J. et al. (2004). Angiogenic activity of human CC chemokine CCL15 in vitro and in vivo. FEBS Letters 570(1-3):47-51.

Iimuro, S. et al. (2004). Angiogenic effects of adrenomedullin in ischemia and tumor growth. Circ Res 95(4):415-423.

Judge, A. D. et al. (2005). Sequence-dependent stimulation of the mammalian innate immune response by synthetic siRNA. Nat Biotechnol 23:457-462.

Juhasz, A. et al. (2003). Quantification of Chemotherapeutic Target Gene mRNA Expression in Human Breast Cancer Biosies: Comparison of Real-Time Reverse Transcription-PCR vs Relative Quantification Reverse Transcription-PCR Utilizing DNA Sequencer Analysis of PCR Products. J Clin Lab Anal 17:184-194.

Kawahara, T. et al. (2005). Point mutations in the proline-rich region of p22phox are dominant inhibitors of Nox1- and Nox2-dependent reactive oxygen generation. J Biol Chem 280:31859-31869.

Ketting, R. F. et al. (2001). Dicer functions of RNA interference and in synthesis of small RNA involved in developmental timing in C. elegans. Genes Dev 15(20):2654-2659.

Kikuchi, H. et al. (2000). NADPH oxidase subunit, gp91^{phox} homologue, preferentially expressed in human colon epithelial cells. Gene 254:237-243.

Kim, D. H. et al. (2005). Synthetic dsRNA Dicer substrates enhance RNAi potency and efficacy. Nat Biotechnol 23:222-226.

Kim, E.-J. et al. (2005). IFI16 is an Essential Mediateor of Growth Inhibition, but Not Differentiation, Induced by the Leukemia Inhibitory Factor/JAK/Stat Pathway in Medullary Thyroid Carcinoma Cells. J Biol Chem 280(6):4913-4920.

Knies-Bamforth, U. E. et al. (2004). c-Myc Interacts with Hypoxia to Induce Angiogenesis In vivo by a Vascular Endothelial Growth Factor-Dependent Mechanism. Can Res 64:6563-6570.

Kreuter, J. (1991). Nanoparticles-preparation and applications. In: Microcapsules and nanoparticles in medicine and pharmacy, Donbrow M., ed, CRC Press, Boca Raton, Fla., pp. 125-14.

Kuwano, Y. et al. (2006). Interferon-gamma activates transcription of NADPH oxidase I gene and regulates production of superoxide anion by human large intestinal epithelial cells. Am J Physiol Cell Physiol 290:C433-C443.

Lambeth, J. D. (2002). Nox/Duox family of nicotinamide adenine dinucleotide (phosphate) oxidases. Current Opinion in Hematology 9:11-17.

Lambeth, J. D. (2004). NOX enzymes and the biology of reactive oxygen. Nature Reviews/Immunology 4:181-189.

Lambeth, J. D. et al., (2000). Novel homologs of gp91phox. TIBS 25:459-461.

Lassegue, B. et al. (2001). Novel gp91(phox) homologues in vascular smooth muscle cells: nox1 mediates angiotensin II-induced superoxide formation and redox-sensitive signaling pathways. Circ Res 88(9):888-94.

Lee, N. S. et al. (2002). Expression of small interfering RNAs targeted against HIV-1 rev transcripts in human cells. Nat Biotechnol 20(5):500-505.

Lee, Y. S. et al. (2004). Distinct roles for Drosophila Dicer-1 and Dicer-2 in the siRNA/miRNA silencing pathways. Cell 117:69-81.

Lei, W. et al. (2005). Positive and negative regulation of c-Myb by cyclin D1, cyclin-dependent kinases, and p27Kip1. Blood 105(10):3855-3861.

Li, Q. and Engelhardt, J. F. (2006). Interleukin-1beta Induction of NFkB Is Partially regulated by H2O2-mediated Activation of NFkB-inducing Kinase. J Biol Chem 281(3):1495-1505.

Lim, S. D. et al. (2005). Increased Nox1 and hydrogen peroxide in prostate cancer. Prostate 62(2):200-7.

Ma, Z. et al. (2005). Cationic lipids enhance siRNA-mediated interferon response in mice. Biochem Biophys Res Commun 330:755-759.

Menon, S. G. et al. (2003). Redox regulation of the G1 to S Phase Transition in the Mouse Embryo Fibroblast Cell Cycle. Can Res 63:2109-2117.

Mezquita, P. et al. (2005). Myc regulates VEGF production in B cells by stimulating initiation of VEGF mRNA translation. Oncogene 24:889-901.

Mitsushita, J. et al. (2004). The Superoxide-Generating Oxidase Nox1 is Functionally Requiredfor Ras Oncogene Transformation. Can Res 64:3580-3585.

Miyagishi, M. and Taira, K. (2002). U6 promoter driven siRNAs with four uridine 3′ overhangs efficiently suppress targeted gene expression in mammalian cells. Nat Biotechnol 20(5):497-500.

Moss, E. G. (2001). RNA interference: it's a small RNA world. Curr Biol 11(19):R772-R775.

North, S. et al. (2005). Recent developments in the regulation of the angiogenic switch by cellular stress factors in tumors. Cancer Letters 218:1-14.

Ottaiano A. et al. (2005). Inhibitory effects of anti-CXCR4 antibodies on human colon cancer cells. Cancer Immunol Immunother 54:781-791.

Park, H. S. et al. (2004). Sequential Activation of Phophatidylinositol 3-kinase, bPix, Rac1, and Nox1 in Growth Factor-Induced Production of H₂O₂. Mol Cell Biol 24(10):4384-4394.

Paul, C. P. et al. (2002). Effective expression of small interfering RNA in human cells. Nat Biotechnol 20(5):505-508.

Perner, A. et al. (2003). Superoxide production and expression of NAD(P)H oxidases by transformed andprimary human colonic epithelial cells. Gut 52:231-236.

Pham, J. W. et al. (2004). A Dicer-2-dependent 80s complex cleaves targeted mRNAs during RNAi in Drosophila. Cell 117:83-94.

Reynolds, A. et al. (2004). Rational siRNA design for RNA interference. Nat Biotechnol 22:326-330.

Rose, S. D. et al. (2005). Functional polarity is introduced by Dicer processing of short substrate RNAs. Nucleic Acids Research 33(13):4140-4156.

Rusckowski, M. et al. (2000). Biodistribution and metabolism of a mixed backbone oligonucleotide (GEM 231) following single and multiple dose administration in mice. Antisense Nucleic Acid Drug Dev 10:333-345.

Saccani, A. et al. (2000). Redox regulation of chemokine receptor expression. Proc Natl Acad Sci USA 97(6):2761-2766.

Schioppa T. et al. (2003). Regulation of the chemokine receptor CXCR4 by hypoxia. J Exp Med 198(9):1391-1402.

Semenza, G. L. (2003). Targeting HIF-1 for cancer therapy. Nature Reviews/Cancer 3:721-732.

Sherr, C. et al., (1999). CDK inhibitors: positive and negative regulators of G1-phase progression. Genes & Development 13:1501 - 1512.

Shridhar R. et al. (2002). Cystatin M suppresses the malignant phenotype of human MDA-MB-435S cells. Oncogene 23(12):2206-2216.

Siolas, D. et al. (2005). Synthetic shRNAs as potent RNAi triggers. Nat. Biotechnol 23:227-231.

Sledz, C. A. et al. (2003). Activation of the interferon system by short-interfering RNAs. Nature Cell Biol 5:834-839.

Sontheimer, E. J. (2005). Assembly and function of RNA silencing complexes. Nature Rev Mol Cell Biol, 6:127-138.

Sorescu, D. et al. (2002). Superoxide production and expression of nox family proteins in human atherosclerosis. Circulation 105(12):1429-1435.

Stein, D. A. et al. (2001). Inhibition of Vesivirus infections in mammalian tissue culture with antisense morpholino oligomers. Antisense Nucleic Acid Drug Dev 11:317-25.

Suh, Y.-A. et al. (1999). Cell transformation by superoxide-generating oxidase Mox1. Nature 401:79-82.

Szanto, I. et al. (2005). Expression of NOX1, a superoxide-generating NADPH oxidase, in colon cancer and inflammatory bowel disease. J Pathology 207:164-176.

Szatrowski, T. P. and Nathan, C. F. (1991). Production of Large Amounts of Hydrogen Peroxide by Human Tumor Cells. Cancer Research 51:794-798.

Takeya, R. et al. (2003). Novel human homologues of p47phox and p67phox participate in activation of superoxide-producing NADPH oxidases. J Biol Chem 278(27):25234-25246.

Tomari, Y. et al. (2004). A protein sensor for siRNA asymmetry. Science 306:1377-1380.

Verma, S. and Eckstein, F. (1998). Modified oligonucleotides: synthesis and strategy for users. Annu Rev Biochem 67:99-134.

Vermeulen, A. et al. (2005). The contributions of dsRNA structure to Dicer specificity and efficiency. RNA 11:674-682.

Vorobjev, P. E. et al. (2001). Nuclease resistance and RNase H sensitivity of oligonucleotides bridged by oligomethylenediol and oligoethylene glycol linkers. Antisense Nucleic Acid Drug Dev 11:77-85.

Wingler, K. et al. (2001). Upregulation of the vascular NAD(P)H-oxidase isoforms Nox1 and Nox4 by the renin-angiotensin system in vitro and in vivo. Free Radic Biol Med 31(11):1456-1464.

Yu, J. Y. et al. (2002). RNA interference by expression of short-interfering RNAs and hairpin RNAs in mammalian cells. Proc Natl Acad Sci USA 99(9):6047-6052.

Zhang, H. S. et al. (1999). Active Transcriptional Repression by the RB-E2F Complex Mediates G1 Arrest Triggered by p16INK4, TGFbeta and Contact Inhibition. Cell 97:53-61.

What is claimed is:

1. A small interfering RNA (siRNA), comprising a sense strand and an antisense strand, wherein the antisense strand has a sequence sufficiently complementary to a NOS1 target sequence to direct target-specific RNA interference (RNAi)

2. The siRNA of claim 1, wherein each strand of the siRNA molecule comprises about 14 to about 30 nucleotides, and wherein each strand comprises at least about 14 to 27 nucleotides that are complementary to the nucleotides of the other strand.

3. The siRNA of claim 1, wherein the siNA is assembled from two separate oligonucleotide fragments wherein a first fragment comprises the sense strand and a second fragment comprises the antisense strand of said siNA molecule.

4. The siRNA of claim 3, wherein the sense strand is connected to the antisense strand via a linker molecule.

5. The siRNA of claim 4, wherein the linker molecule is a polynucleotide linker or a non-nucleotide linker.

6. The siRNA of claim 1, wherein the siRNA comprises a modification.

7. The siRNA of claim 6, wherein the modification is selected from the group consisting of:
(a) cap moiety at a 5′-end, a 3′-end, or both, (b) phosphorothioate internucleotide linkage at the 3′ end, (c) at least one 2′-sugar modification, (d) at least one nucleic acid base modification, (e) at least one phosphate backbone modification and (f) any combination of (a)-(e).

8. The siRNA of claim 1, wherein the target sequence is selected from the group consisting of SEQ ID NO:1, SEQ ID NO:2, SEQ ID NO:3 and SEQ ID NO:4.

9. A vector comprising the siRNA of claim 1.

10. A pharmaceutical composition comprising the siRNA of claim 1 or the vector of claim 9 and a pharmaceutically acceptable carrier.

11. A pharmaceutical composition comprising the vector of claim 9 and a pharmaceutically acceptable carrier.

12. A method of activating target-specific RNA interference (RNAi) in a colon cancer cell comprising introducing into said cell the siRNA of claim 1, the siRNA being introduced in an amount sufficient for degradation of the NOS1 target sequence to occur, thereby activating target-specific RNAi in the cell.

13. The method of claim 12, wherein the siRNA is introduced into the cell by contacting the cell with the siRNA.

14. The method of claim 12, wherein the siRNA is introduced into the cell by contacting the cell with a composition comprising the siRNA and a lipophillic carrier.

15. The method of claim 12, wherein the siRNA is introduced into the cell by transfecting or infecting the cell with a vector comprising nucleic acid sequences capable of producing the siRNA when transcribed in the cell.

16. The method of claim 12, wherein the siRNA is introduced into the cell by injecting into the cell a vector comprising nucleic acid sequences capable of producing the siRNA when transcribed in the cell.

17. The method of claim 12, wherein the colon cancer cell is of mammalian origin.

18. The method of claim 17, wherein the cell is of human origin.


SEQUENCE LISTING (8 sequences)

SEQ ID NO: 1 - DNA, 21 bp, Homo sapiens
gagaaggccgacaaatactac

SEQ ID NO: 2 - DNA, 21 bp, Homo sapiens
gctcattttgcagccgcacac

SEQ ID NO: 3 - DNA, 21 bp, Homo sapiens
gagatgtgggatgatcgtgac

SEQ ID NO: 4 - DNA, 21 bp, Homo sapiens
ggctttcgaacaacaatattc

SEQ ID NO: 5 - DNA, 21 bp, Artificial
  scrambled control sequence
gtcgtaccgtacgtaagacac

SEQ ID NO: 6 - DNA, 326 bp, Artificial
  DNA construct encoding shRNA
ggatccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcgtaccgtacgtaagacactttgtgtagtgtcttacgtacggtacgactttttt

SEQ ID NO: 7 - DNA, 326 bp, Artificial
  DNA construct encoding shRNA
ggatccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcacgatcatcccacatctctttgtgtagagatgtgggatgatcgtgactttttt

SEQ ID NO: 8 - DNA, 326 bp, Artificial
  DNA construct encoding shRNA
ggatccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaatattgttgttcgaaagcctttgtgtaggctttcgaacaacaatattctttttt